(19) United States
(12) Patent Application Publication
(10) Pub. No.: US 2026/0272797 A1
(43) Pub. Date: Sep. 17, 2026

(54) FRAGRANCE COMPOSITION

(71) Applicant: GIVAUDAN SA, Vernier (CH)

(72) Inventor: Elisabeth RINAUDO, Argentuil (FR)

(21) Appl. No.: 19/472,265

(22) PCT Filed: Apr. 9, 2024
(86) PCT No.: PCT/EP2024/059604

§ 371 Date: Oct. 3, 2025

(30) Foreign Application Priority Data
Apr. 13, 2023 (GB) 2305429.9
Dec. 19, 2023 (GB) 2319461.6

(51) Int. Cl. A61K 8/368; A61K 8/31; A61K 8/33; A61K 8/34; A61K 8/35; A61K 8/36; A61K 8/37; A61K 8/39; A61K 8/44; A61K 8/49; A61K 8/92; A61K 8/9789; A61Q 13/00; A61Q 15/00; G01N 33/00

(52) U.S. Cl. CPC A61K 8/368; A61K 8/31; A61K 8/33; A61K 8/342; A61K 8/347; A61K 8/35; A61K 8/361; A61K 8/37; A61K 8/39; A61K 8/445; A61K 8/4973; A61K 8/498; A61K 8/922; A61K 8/9789; A61Q 13/00; A61Q 15/00; G01N 33/0001; A61K 2800/591

(57) ABSTRACT

The present disclosure provides a fragrance composition adapted to mask, reduce and/or prevent malodours, more particularly scalp malodour, to a method for assessing scalp malodour counteractancy, to a consumer product comprising said fragrance composition and to a method for masking, reducing and/or preventing scalp malodours.

The present invention generally relates to a fragrance composition adapted to mask, reduce and/or prevent malodours, more particularly scalp malodour, to a method for assessing scalp malodour counteractancy, to a consumer product comprising said fragrance composition and to a method for masking, reducing and/or preventing scalp malodours.

It is well known that most body malodours are generated by the breakdown of secretions by bacteria present on the skin. In particular, scalp malodour typically occurs when the hair is covered by a fabric over an extended period of time. This malodour generation is furthermore favoured by hot and humid climates. Typically, the fabric is a cotton, a polyester or a cotton-polyester fabric. The presence of polyester is an additional factor of malodour production, owing to the lower breathability of this material.

The scalp malodour is endogenous and generated by the scalp itself. Scalp malodour is different from other malodours that may develop on the hair itself and which are exogenous, i.e. adsorbed from external sources, such as kitchen, smoke or gasoil pollution. The components of the scalp malodour are mostly carboxylic acids and particularly C5 to C13 aldehydes, C3 to C11 methyl ketones and C4 to C9 gamma lactones, whereas exogenous malodours typically contain sulphur-and/or nitrogen-containing compounds.

A common way to counteract (exogenous) hair malodour is to use shampoos that combine the cleaning effect of surfactants, sometimes in combination with anti-dandruff agents, as well as the malodour masking, reducing and/or preventing effect of fragrance ingredients.

However, the prior art remains silent as to how to counteract the specific malodour emanating from the scalp itself. In particular, the possibility to use certain fragrance compositions to mask, reduce and/or prevent specifically scalp malodour has not been contemplated so far.

The present invention is based on the surprising discovery that certain fragrance ingredients provide efficient scalp malodour counteraction, whereas, on the contrary, other fragrance ingredients enhance scalp malodour.

Therefore, in a first aspect, the present invention provides a malodour counteracting fragrance composition, which can be used to mask, reduce and/or prevent scalp malodour.

In a second aspect, the present invention provides a method for assessing the scalp malodour counteraction capability of a test fragrance composition.

In a third aspect, the present invention provides a cosmetic product comprising the scalp malodour counteracting fragrance composition of the present invention.

In a fourth aspect, the present invention provides a method of masking, reducing and/or preventing body malodour, in particular scalp malodour, said method comprising the step of topically applying the malodour counteracting fragrance composition or the cosmetic product of the present invention to the human body, in particular to the hair or scalp.

In the context of the present disclosure, the term “fragrance composition” relates to a mixture of fragrance ingredients, including if desired a suitable solvent or solvents and optionally also mixed with a solid substrate.

Fragrance ingredients are well known to those skilled in the art, and include those mentioned, for example, in S. Arctander, Perfume and Flavor Chemicals (Montclair, N.J., 1969), in S. Arctander, Perfume and Flavor Materials of Natural Origin (Elizabeth, N.J., 1960) and in “Flavor and Fragrance Materials-1991”, Allured Publishing Co. Wheaton, III. USA. Fragrance ingredients may include natural products, such as extracts, essential oils, absolutes, resinoids, resins, concretes, etc., and also synthetic substances such as hydrocarbons, alcohols, aldehydes, ketones, ethers, acids, esters, acetals, ketals, nitriles, etc., including saturated and unsaturated compounds, aliphatic, carbocyclic and heterocyclic compounds.

In the context of the present disclosure, the term “reconstitution” relates to a mixture of fragrance ingredients having an odour that is similar to the odour of a natural product. For example, the smell of a “blackcurrant reconstitution” smells similar or is reminiscent of the smell of a blackcurrant oil. A reconstitution may or may not contain the same or similar fragrance ingredients as those contained in the natural product.

In the context of the present disclosure, the term “malodour counteracting fragrance composition” relates to a composition, which is able to mask, reduce and/or prevent the perception of a malodour. Such malodour counteracting fragrance compositions may be identified by the method of the present invention disclosed below, or by similar methods known from the prior art.

The scalp malodour counteracting fragrance composition of the present invention comprises one or more fragrance ingredients drawn from Group A and/or one or more fragrance ingredients drawn from Group A and one or more fragrance ingredients drawn from Group B, wherein the sum of the individual concentrations of all fragrance ingredients drawn from Group A is 10 wt. % or more, based on the total weight of the scalp malodour counteracting fragrance composition; wherein the sum of the individual concentrations of all fragrance ingredients drawn from Group B is 0.1 wt. % or more, based on the total weight of the scalp malodour counteracting fragrance composition; and wherein the weight ratio of the sum of the individual concentrations of all fragrance ingredients drawn from Group A to the sum of the individual concentrations of all fragrance ingredients drawn from Group B is 0.8 or more.

According to the invention, the Group A fragrance ingredients are selected from the group consisting of 3-hydroxybutan-2-one (e.g. ACETOIN); 1-phenylethanone (e.g. ACETOPHENONE); 2-(tert-butyl)cyclohexyl acetate (e.g. AGRUMEX); 2-methylundecanal (e.g. ALDEHYDE C 12 MNA PURE); 1-((2-(tert-butyl)cyclohexyl)oxy) butan-2-ol (e.g. AMBER CORE); (4aR,5R,7aS,9R)-octahydro-2,2,5,8,8,9a-hexamethyl-4H-4a,9-methanoazuleno [5,6-d]-1,3-dioxole (e.g. AMBRO-CENIDE); (3aR,5aS,9aS,9bR)-3a,6,6,9a-tetramethyl-2,4,5,5a,7,8,9,9b-octahydro-1H-benzo[e][1]benzofuran (e.g. AMBROFIX); (E)-1-methoxy-4-(prop-1-en-1-yl)benzene (e.g. ANETHOLE); anise and star anise oils; 4-methoxybenzaldehyde (e.g. AUBEPINE PARA CRESOL); 7-isopentyl-2H-benzo[b][1,4]dioxepin-3 (4H)-one (e.g. AZURONE); benzoin oils; 1-butoxy-1-oxopropan-2-yl butanoate (e.g. BUTYL BUTYRO LACTATE); 8-methyl-1,5-benzodioxepin-3-one (e.g. CALONE); 6-methoxy-2,6-dimethyloctanal (e.g. CALYPSONE); (1S,4S)-1,7,7-trimethylbicyclo[2.2.1]heptan-2-one (e.g. CAMPHOR); blackcurrant oils and reconstitutions; 5-tert-butyl-2-methyl-5-propyl-2H-furan (e.g. CASSYRANE); cedar wood oils; (1S,6R,8aR)-1,4,4,6-tetramethyloctahydro-1H-5,8a-methanoazulen-6-yl acetate (e.g. CEDRYL ACETATE); 3,7-dimethyloct-6-enal (e.g. CITRONELLAL); 3,7-dimethyloct-6-en-1-ol (e.g. CITRONELLOL); clove oils and reconstitutions (4-isopropylphenyl) methanol (e.g. CUMINYL ALCOHOL); cyclohexyl 2-hydroxybenzoate (e.g. CYCLOHEXYL SALICYLATE); (E)-1-(2,6,6-trimethylcyclohexa-1,3-dien-1-yl) but-2-en-1-one (e.g. DAMASCENONE); (E)-1-(2,6,6-trimethylcyclohex-2-en-1-yl) but-2-en-1-one (e.g. DAMASCONE ALPHA); (E)-1-(2,6,6-trimethyl-1-cyclohexenyl) but-2-en-1-one (e.g. DAMASCONE BETA); 5-hexyloxolan-2-one (e.g. DECALACTONE GAMMA); 2-methoxy-4-propylphenol (e.g. DIHYDRO EUGENOL); 6-heptyltetrahydro-2H-pyran-2-one (e.g. DODECALACTONE DELTA); (E)-4-((3aS,7aS)-hexahydro-1H-4,7-methanoinden-5 (6H)-ylidene) butanal (e.g. DUPICAL); (E)-3-methyl-5-(2,2,3-trimethylcyclopent-3-en-1-yl) pent-4-en-2-ol (e.g. EBANOL); ethyl 2-methylpropanoate (e.g. ETHYL ISOBUTYRATE); ethyl octanoate (e.g. ETHYL OCTANOATE); ethyl heptanoate (e.g. ETHYL OENANTHATE); 4-allyl-2-methoxyphenol (e.g. EUGENOL); 3-(4-methoxyphenyl)-2-methylpropanal (e.g. FENNALDEHYDE); 1-(3,5,5,6,8,8-hexamethyl-5,6,7,8-tetrahydronaphthalen-2-yl) ethanone (e.g. FIXOLIDE); ethyl 2-(2-methyl-1,3-dioxolan-2-yl)acetate (e.g. FRUCTONE); 4,6,6,7,8,8-hexamethyl-1,3,4,6,7,8-hexahydrocyclopenta [g]isochromene (e.g. GALAXOLIDE); geranium oils; benzo[d][1,3]dioxole-5-carbaldehyde (e.g. HELIOTROPIN; 7-hydroxy-3,7-dimethyloctanal (e.g.); 3,7-dimethyloctane-1,7-diol (e.g. HYDROXYCITRONELLOL); 1H-indole (e.g. INDOLE PURE); 1-(2,3,8,8-tetramethyl-1,2,3,4,5,6,7,8-octahydronaphthalen-2-yl) ethanone (e.g. ISO E SUPER); 4-formyl-2-methoxyphenyl 2-methylpropanoate (e.g. ISOBUTAVAN); (E)-2-methoxy-4-(prop-1-en-1-yl) phenol (e.g. ISOEUGENOL); (Z)-1-(2,6,6-trimethylcyclohex-2-en-1-yl) pent-1-en-3-one, (E)-1-(2,6,6-trimethylcyclohex-2-en-1-yl) pent-1-en-3-one and mixtures thereof (e.g. ISORALDEINE); (E)-3-methyl-4-(2,6,6-trimethylcyclohex-2-en-1-yl) but-3-en-2-one (e.g. ISORALDEINE CETONE ALPHA); (1-methyl-2-((1,2,2-trimethylbicyclo[3.1.0]hexan-3-yl)methyl)cyclopropyl) methanol (e.g. JAVANOL); 3-(4-(tert-butyl)phenyl)-2-methylpropanal (e.g. LILIAL); (4E)-9-hydroxy-5,9-dimethyl-4-decenal (e.g. MAHONIAL); 3-hydroxy-2-methyl-4H-pyran-4-one (e.g. MALTOL); (4-isopropyl-cyclohexyl) methanol (e.g. MAYOL); 3-methyl-5-phenylpentan-1-ol (e.g. MEFROSOL); 2-isopropyl-5-methylcyclohexanol (e.g. MENTHOL); mercapto-para-menthan-3-one (e.g. MERCAPTO-8 P-MENTHANE-3-ONE); methyl 2-aminobenzoate (e.g. METHYL ANTHRANILATE); 8-methyl-1-oxaspiro[4.5]decan-2-one (e.g. METHYL LAITONE); 8-ethyl-1-oxaspiro[4.5]decan-2-one (e.g. ETHYL LAITONE); methyl non-2-ynoate (e.g. METHYL OCTYNE CARBONATE); (E)-3-methyl-4-(2,6,6-trimethylcyclohex-1-en-1-yl) but-3-en-2-one (e.g. METHYLIONANTHEME); (Z)-3-methylcyclopentadec-5-enone (e.g. MUSCENONE); (E)-methyl non-2-enoate (e.g. NEOFOLIONE); (2Z)-3,7-dimethylocta-2,6-dien-1-ol (e.g. NEROLEX); (E)-3,7,11-trimethyldodeca-1,6,10-trien-3-ol (e.g. NEROLIDOL); (E)-13-methyloxacyclopentadec-10-en-2-one (e.g. NIRVANOLIDE); 2-(6,6-dimethylbicyclo[3.1.1]hept-2-en-2-yl)ethyl acetate (e.g. NOPYL ACETATE); 3-(4-(2-methylpropyl)-2-methylphenyl) propanal (e.g. NYMPHEAL); patchouli oils; 3,7-dimethyloctan-1-ol (e.g. PELARGOL); (2E,5E)-5,6,7-trimethylocta-2,5-dien-4-one (e.g. POMAROSE); (2R,8aS)-3′,6-dimethyl-3,4,4a,5,8,8a-hexahydro-1H-spiro[1,4-methanonaphtha-lene-2,2′-oxirane]); 1-methyl-2-(5-methylhex-4-en-2-yl)cyclopropylmethanol (e.g. ROSYFOLIA); 2-(4-methyl-1,3-thiazol-5-yl) ethanol (e.g. SULFUROL); (E)-6-ethyl-3-methyloct-6-en-1-ol (e.g. SUPER MUGUET); 1-((2E,5Z,9Z)-2,7,8-trimethylcyclododeca-2,5,9-trien-1-yl) ethanone (e.g. TRIMOFIX O); 2-ethoxy-4-methylphenol (e.g. ULTRAVANIL); 2,2,5-trimethyl-5-pentylcyclo-pentanone (e.g. VELOUTONE); and mixture thereof.

According to the invention, the Group B fragrance ingredients are selected from the group consisting of (2-benzyl-1,3-dioxolan-4-yl) methanol (e.g. ACETAL CD); undecanal (e.g. ALDEHYDE C 110 UNDECYLIC); prop-2-enyl 3-cyclohexylpropanoate (e.g. ALLYL CYCLOHEXYL PROPIONATE); pineapple oils and reconstitutions; benzyl acetate; benzyl propionate; (5R)-2-methyl-5-prop-1-en-2-ylcyclohex-2-en-1-one (e.g. CARVONE); (E)-1-(2,6,6-trimethylcyclohex-2-en-1-yl) hepta-1,6-dien-3-one (e.g. CETONE V); 3,7-dimethyloct-6-en-1-yl acetate (e.g. CITRONELLYL ACETATE); 2,4-dimethylcyclohex-3-ene-1-carbaldehyde (e.g. CYCLAL C); allyl 2-(cyclohexyloxy)acetate (e.g. CYCLOGALBANATE); 4-(2,6,6-trimethylcyclohex-1-en-1-yl) butan-2-one (e.g. DIHYDRO IONONE BETA); 2-methyl-1-phenylpropan-2-ol (e.g. DIMETHYL BENZYL CARBINOL); 2-methyl-1-phenylpropan-2-yl acetate (e.g. DIMETHYL BENZYL CARBINYL ACETATE); 2-methyl-1-phenylpropan-2-yl butanoate (e.g. DIMETHYL BENZYL CARBINYL BUTYRATE); 4,7-dimethyloct-6-en-3-one (e.g. DIMETHYL OCTENONE); (2E,4Z)-ethyl deca-2,4-dienoate (e.g. ETHYL DECADIENOATE); ethyl 2,6,6-trimethylcyclohexa-1,3-diene-1-carboxylate (e.g. ETHYL SAFRANATE); 4-allyl-2-methoxyphenyl acetate (e.g. EUGENYL ACETATE); (3aR,6S,7aS)-3a,4,5,6,7,7a-hexahydro-1H-4,7-methanoinden-6-yl propanoate (e.g. FLOROCYCLENE); 2,4,6-trimethyl-4-phenyl-1,3-dioxane (e.g. FLOROPAL); tetrahydro-4-methyl-2-(2-methylpropyl)-2H-pyran-4-ol (e.g. FLOROSA and FLOROSA HC); 2-ethyl-4-hydroxy-5-methylfuran-3 (2H)-one (e.g. HOMOFURONOL); (Z)-hex-3-en-1-yl benzoate (e.g. HEXENYL-3-CIS BENZOATE); (E)-2-benzylideneoctanal (e.g. HEXYL CINNAMIC ALDEHYDE); (E)-4-(2,6,6-trimethylcyclohex-1-en-1-yl) but-3-en-2-one (e.g. IONONE BETA); (E)-4-(2,6,6-trimethyl-1-cyclohex-2-enyl) but-3-en-2-one (e.g. IRISONE ALPHA, IRISONE PURE or IONONE ALPHA); (3aR,6S,7aS)-3a,4,5,6,7,7a-hexahydro-1H-4,7-methanoinden-6-yl acetate (e.g. JASMACYCLENE); (Z)-3-methyl-2-(pent-2-en-1-yl)cyclopent-2-enone (e.g. JASMONE CIS); 3-butyl-5-methyltetrahydro-2H-pyran-4-yl acetate (e.g. JASMONYL); lemongrass oils; lime oils; 1-((1S,8aS)-1,4,4,6-tetramethyl-2,3,3a,4,5,8-hexahydro-1H-5,8a-methanoazulen-7-yl) ethanone (e.g. METHYL CEDRYL KETONE); methyl 2-hexyl-3-oxocyclopentane-1-carboxylate (e.g. METHYL DIHYDRO ISOJASMONATE); (E)-1,2-dimethoxy-4-(prop-1-en-1-yl)benzene (e.g. METHYL ISOEUGENOL); 4-(4-methylpent-3-en-1-yl)cyclohex-3-enecarbaldehyde (e.g. MYRALDENE); (Z)-3,7-dimethylocta-2,6-dien-1-yl acetate (e.g. NERYL ACETATE); 6-propyltetrahydro-2H-pyran-2-one (e.g. OCTALACTONE DELTA); 5-butyloxolan-2-one (e.g. OCTALACTONE GAMMA); blood orange oils; orange oils; bitter orange oils; orange terpenes (e.g. terpene rich fraction of orange oil); sweet orange oils; 1-(2-naphtalenyl)-ethanone (e.g. ORANGER CRYSTALS); 2-methyl-4-propyl-1,3-oxathiane (e.g. OXANE); passion fruit oils; 2-cyclohexylidene-2-phenylacetonitrile (e.g. PEONILE); 2-(phenoxy)ethyl 2-methylpropanoate (e.g. PHENOXY ETHYL ISOBUTYRATE); 2-phenylethyl acetate (e.g. PHENYL ETHYL ACETATE); 2-phenylethanol (e.g. PHENYL ETHYL ALCOHOL); 2,4-dimethyl-4-phenyl-tetrahydrofuran (e.g. RHUBAFURAN); cyclopentadecanone, hexadecanolide (e.g. SILVANONE SUPRA); spearmint oils; ethyl methyl phenyl glycidate (e.g. STRAWBERRY PURE); (E)-2-((3,5-dimethylhex-3-en-2-yl)oxy)-2-methylpropyl cyclopropanecarboxylate (e.g. SYLKOLIDE); 3,7-dimethyloctan-3-ol (e.g. TETRAHYDRO LINALOOL); 2,4-dimethylcyclohex-3-enecarbaldehyde (e.g. TRICYCLAL); (Z)-cyclohexadec-5-enone (e.g. VELVIONE); and mixtures thereof.

Throughout this disclosure, concentrations of fragrance ingredients are indicated in wt. % based on the total weight of the scalp malodour counteracting fragrance composition, if not indicated otherwise. Furthermore, throughout this disclosure, the scalp malodour counteracting fragrance composition may include fragrance ingredients, solvents and other additives, such as solubilizing agents, antioxidants, and chelating agents. The total weight of the scalp malodour counteracting fragrance composition is therefore the sum of the weights of the individual fragrance ingredients, of the individual solvents and of the additives.

Surprisingly, it has been found that by carefully selecting and combining certain fragrance ingredients according to the above specified rules, it is possible to provide a highly effective scalp malodour counteracting fragrance composition. More particularly, it has been found that Group A ingredients have the capability to mask, reduce and/or prevent scalp malodours, whereas Group B fragrance ingredients have the tendency to enhance the scalp malodour or to distort this malodour in an undesirable way, but are overall still desirable from a hedonic point of view. The selection is generally performed by maximizing the level of Group A fragrance ingredients and minimizing the level of Group B ingredients.

The malodour counteractancy was confirmed by sensory studies, an example of which is described below.

It was also found that, within the Group A fragrance ingredients, not all ingredients are equivalent in their capability to mask, reduce and/or prevent scalp malodours. For example, certain ingredients are more effective scalp malodour counteractants at a lower or a higher concentration than other ingredients. Therefore, the Group A ingredients may be classified in three sub-groups, referred to as Group A1, Group A2 and Group A3 ingredients hereinafter.

Independently from this, fragrance compositions with a larger number of different fragrance ingredients tend to be perceived as more structured, more complex and overall more hedonically pleasing. Hence, a larger number of different fragrance ingredients is generally desirable.

In an embodiment, the scalp malodour counteracting fragrance composition according to the present invention comprises, based on the total weight of the scalp malodour counteracting fragrance composition, at least 8 wt. %, preferably at least 10 wt. %, of at least 1, preferably at least 2, and more preferably at least 3 Group A fragrance ingredients drawn from Group A1, wherein the Group A1 fragrance ingredients are selected from the group consisting of 2-(tert-butyl)cyclohexyl acetate (e.g. AGRUMEX); 4-methoxybenzaldehyde (e.g. AUBEPINE PARA CRESOL); benzoin oils; 3,7-dimethyloct-6-en-1-ol (e.g. CITRONELLOL); 5-hexyloxolan-2-one (e.g. DECALACTONE GAMMA); ethyl 2-(2-methyl-1,3-dioxolan-2-yl)acetate (e.g. FRUCTONE); 4,6,6,7,8,8-hexamethyl-1,3,4,6,7,8-hexahydrocyclopenta [g]isochromene (e.g. GALAXOLIDE); benzo[d][1,3]dioxole-5-carbaldehyde (e.g. HELIOTROPINE); 7-hydroxy-3,7-dimethyloctanal (e.g. HYDROXYCITRONELLAL); 3,7-dimethyloctane-1,7-diol (e.g. HYDROXYCITRONELLOL); 1-(2,3,8,8-tetramethyl-1,2,3,4,5,6,7,8-octahydronaphthalen-2-yl) ethanone (e.g. ISO E SUPER (Z)-1-(2,6,6-trimethylcyclohex-2-en-1-yl) pent-1-en-3-one, (E)-1-(2,6,6-trimethylcyclohex-2-en-1-yl) pent-1-en-3-one and mixtures thereof (e.g. ISORALDEINE); 3-(4-(tert-butyl)phenyl)-2-methylpropanal (e.g. LILIAL); (4-isopropylcyclohexyl) methanol (e.g. MAYOL); 2-(6,6-dimethylbicyclo[3.1.1]hept-2-en-2-yl)ethyl acetate (e.g. NOPYL ACETATE); 3-(4-(2-methylpropyl)-2-methylphenyl) propanal (e.g. NYMPHEAL); 1-methyl-2-(5-methylhex-4-en-2-yl)cyclopropylmethanol (e.g. ROSYFOLIA); 1-((2E,5Z,9Z)-2,7,8-trimethylcyclododeca-2,5,9-trien-1-yl) ethanone (e.g. TRIMOFIX O); and mixtures thereof.

In an embodiment, the scalp malodour counteracting fragrance composition according to the present invention comprises, based on the total weight of the scalp malodour counteracting fragrance composition, from 0.3 wt. % to 10 wt. %, preferably from 0.7 wt. % to 7 wt. %, and more preferably from 0.9 wt. % to 5 wt. % of at least 1, preferably at least 2, preferably at least 3 Group A fragrance ingredients drawn from Group A2, wherein the Group A2 fragrance ingredients are selected from the group consisting of 3-hydroxybutan-2-one (e.g. ACETOIN); 2-methylundecanal (e.g. ALDEHYDE C 12 MNA PURE); 1-((2-(tert-butyl)cyclohexyl)oxy) butan-2-ol (e.g. AMBER CORE); (3aR,5aS,9aS,9bR)-3a,6,6,9a-tetramethyl-2,4,5,5a,7,8,9,9b-octahydro-1H-benzo[e][1]-benzofuran (e.g. AMBROFIX, AMBROXAN or AMBROX); (E)-1-methoxy-4-(prop-1-en-1-yl)benzene (e.g. ANETHOLE); 1-butoxy-1-oxopropan-2-yl butanoate (e.g. BUTYL BUTYRO LACTATE); 8-methyl-1,5-benzodioxepin-3-one (e.g. CALONE); blackcurrant oils and reconstitutions; cedar wood oils; (1S,6R,8aR)-1,4,4,6-tetramethyloctahydro-1H-5,8a-methanoazulen-6-yl acetate (e.g. CEDRYL ACETATE); (E)-1-(2,6,6-trimethylcyclohexa-1,3-dien-1-yl) but-2-en-1-one (e.g. DAMASCENONE); (E)-1-(2,6,6-trimethylcyclohex-2-en-1-yl) but-2-en-1-one (e.g. DAMASCONE ALPHA); 6-heptyltetrahydro-2H-pyran-2-one (e.g. DODECALACTONE DELTA); (E)-4-((3aS,7aS)-hexahydro-1H-4,7-methanoinden-5 (6H)-ylidene) butanal (e.g. DUPICAL); (E)-3-methyl-5-(2,2,3-trimethylcyclopent-3-en-1-yl) pent-4-en-2-ol (e.g. EBANOL); ethyl 2-methylpropanoate (e.g. ETHYL ISOBUTYRATE); 3-(4-methoxyphenyl)-2-methylpropanal (e.g. FENNALDEHYDE); 1-(3,5,5,6,8,8-hexamethyl-5,6,7,8-tetrahydronaphthalen-2-yl) ethanone (e.g. FIXOLIDE); geranium oils; 4-formyl-2-methoxyphenyl 2-methylpropanoate (e.g. ISOBUTAVAN); (E)-3-methyl-4-(2,6,6-trimethylcyclohex-2-en-1-yl) but-3-en-2-one (e.g. ISORALDEINE CETONE ALPHA); (4E)-9-hydroxy-5,9-dimethyl-4-decenal (e.g. MAHONIAL); 3-hydroxy-2-methyl-4H-pyran-4-one (e.g. MALTOL); 3-methyl-5-phenylpentan-1-ol (e.g. MEFROSOL); 2-isopropyl-5-methylcyclohexanol (e.g. MENTHOL); methyl 2-aminobenzoate (e.g. METHYL ANTHRANILATE); (E)-3-methyl-4-(2,6,6-trimethylcyclohex-1-en-1-yl) but-3-en-2-one (e.g. METHYLIONANTHEME); (Z)-3-methylcyclopentadec-5-enone (e.g. MUSCENONE); (E)-13-methyloxacyclopentadec-10-en-2-one (e.g. NIRVANOLIDE); patchouli oils; 3,7-dimethyloctan-1-ol (e.g. PELARGOL); (2R,8aS)-3′,6-dimethyl-3,4,4a,5,8,8a-hexahydro-1H-spiro[1,4-methano-naphthalene-2,2′-oxirane] (e.g. RHUBOFIX); and mixtures thereof.

In an embodiment, the scalp malodour counteracting fragrance composition according to the present invention comprises, based on the total weight of the malodour counteracting fragrance composition, from 0.05 wt. % to 2 wt. %, preferably from 0.07 wt. % to 1.5 wt. %, and more preferably from 0.1 wt. % to 1 wt. %, of at least 1, preferably at least 2, and more preferably at least 3 Group A fragrance ingredients drawn from Group A3, wherein the Group A3 fragrance ingredients are selected from the group consisting of 1-phenylethanone (e.g. ACETOPHENONE); (4aR,5R,7aS,9R)-Octahydro-2,2,5,8,8,9a-hexamethyl-4H-4a,9-methanoazuleno [5,6-d]-1,3-dioxole (e.g. AMBROCENIDE); anise and star anise oils; 7-isopentyl-2H-benzo[b][1,4]dioxepin-3 (4H)-one (e.g. AZURONE; 6-methoxy-2,6-dimethyloctanal (e.g. CALYPSONE); (1S,4S)-1,7,7-trimethylbicyclo[2.2.1]heptan-2-one (e.g. CAMPHOR); blackcurrant oils and reconstructions 5-tert-butyl-2-methyl-5-propyl-2H-furan (e.g. CASSYRANE); (1S,6R,8aR)-1,4,4,6-tetramethyloctahydro-1H-5,8a-methanoazulen-6-yl acetate (e.g. CEDRYL ACETATE); 3,7-dimethyloct-6-enal (e.g. CITRONELLAL NATURAL); (4-isopropylphenyl) methanol (e.g. CUMINYL ALCOHOL); cyclohexyl 2-hydroxybenzoate (e.g. CYCLOHEXYL SALICYLATE); (E)-1-(2,6,6-trimethyl-1-cyclohexenyl) but-2-en-1-one (e.g. DAMASCONE BETA); 2-methoxy-4-propylphenol (e.g. DIHYDRO EUGENOL); ethyl octanoate (e.g. ETHYL OCTANOATE); ethyl heptanoate (e.g. ETHYL OENANTHATE); 4-allyl-2-methoxyphenol (e.g. EUGENOL); 1H-indole (e.g. INDOLE); (E)-2-methoxy-4-(prop-1-en-1-yl) phenol (e.g. ISOEUGENOL); (1-methyl-2-((1,2,2-trimethylbi-cyclo [3.1.0]hexan-3-yl)methyl)cyclopropyl) methanol (e.g. JAVANOL); mercapto-para-menthan-3-one (e.g. MERCAPTO-8 P-MENTHANE-3-ONE); 8-methyl-1-oxaspiro[4.5]decan-2-one (e.g. METHYL LAITONE); 8-ethyl-1-oxaspiro[4.5]decan-2-one (e.g. ETHYL LAITONE); methyl non-2-ynoate (e.g. METHYL OCTYNE CARBONATE); (E)-methyl non-2-enoate (e.g. NEOFOLIONE); (2Z)-3,7-dimethylocta-2,6-dien-1-ol (e.g. NEROLEX); (E)-3,7,11-trimethyldodeca-1,6,10-trien-3-ol (e.g. NEROLIDOL); (2E,5E)-5,6,7-trimethylocta-2,5-dien-4-one (e.g. POMAROSE); 2-(4-methyl-1,3-thiazol-5-yl) ethanol (e.g. SULFUROL); (E)-6-ethyl-3-methyloct-6-en-1-ol (e.g. SUPER MUGUET); 2-ethoxy-4-methylphenol (e.g. ULTRAVANIL); 2,2,5-trimethyl-5-pentylcyclo-pentanone (e.g. VELOUTONE); and mixtures thereof.

Furthermore, it has been found that both a combination of malodour counteractancy and pleasant hedonic character could be achieved when combining Group A1, A2 and A3 ingredients, preferably at the concentrations defined hereinabove.

In an embodiment, the scalp malodour counteracting fragrance composition according to the present invention comprises at least one fragrance ingredient from at least two groups selected from Groups A1, A2, and A3, preferably at least one fragrance ingredient from each of Groups A1, A2 and A3, more preferably in the concentrations defined hereinabove.

In an embodiment, the scalp malodour counteracting fragrance composition according to claim 5 comprises at least one fragrance ingredient from each of A1, A2 and A3, in the concentrations defined hereinabove, wherein the amounts of Groups A1, A2, A3 and B ingredients fulfil the inequality defined by Equation 1:

$$\frac{{W}_{A 1} + 10 {W}_{A 2} + 100 {W}_{A 3}}{{W}_{B}} > 2 \qquad ( Equation 1 )$$

wherein, W_{A1} is the sum of the individual concentrations of all fragrance ingredients drawn from Group A1 fragrance ingredients expressed in wt. %, W_{A2} is the sum of the individual concentrations of all fragrance ingredients drawn from Group A2 fragrance ingredients expressed in wt. %, W_{A3} is the sum of the individual concentrations of all fragrance ingredients drawn from Group A3 fragrance ingredients expressed in wt. %, and W_{B} is the amount of Group B fragrance ingredients, all weight percentage wt. % being based on the total weight of the scalp malodour counteracting fragrance composition.

When the amounts of Groups A1, A2, A3 and B fulfil the condition defined by Equation 1, an optimal balance of scalp malodour counteractancy and pleasant hedonic character is achieved.

The following combinations of fragrance ingredients drawn from Groups A1, A2 and A3 have been found to be particularly well suited for the scalp malodour counteracting fragrance composition of the present invention.

In an embodiment, the scalp malodour counteracting fragrance composition of the present invention comprises:
1-(2,3,8,8-tetramethyl-1,2,3,4,5,6,7,8-octahydronaphthalen-2-yl) ethanone (e.g. ISO E SUPER);
at least one further woody fragrance ingredient selected from the group consisting of (1-methyl-2-((1,2,2-trimethylbicyclo[3.1.0]hexan-3-yl)methyl)cyclopropyl) methanol (e.g. JAVANOL), (E)-3-methyl-5-(2,2,3-trimethylcyclopent-3-en-1-yl) pent-4-en-2-ol (e.g. EBANOL), cedar wood oil, and mixtures thereof;
at least one lactone selected from the group consisting of 5-hexyloxolan-2-one (e.g. DECALACTONE GAMMA), 6-pentyltetrahydro-2H-pyran-2-one (e.g. DECALACTONE DELTA), 5-octyloxolan-2-one (e.g. DODECALACTONE GAMMA), 8-isopropyl-1-oxaspiro[4.5]decan-2-one (e.g. LAITONE), 8-methyl-1-oxaspiro[4.5]decan-2-one (e.g. METHYL LAITONE), 8-ethyl-1-oxaspiro[4.5]decan-2-one (e.g. ETHYL LAITONE), and mixtures thereof;
at least one floral fragrance ingredient selected from the group consisting of (Z)-1-(2,6,6-trimethylcyclohex-2-en-1-yl) pent-1-en-3-one, (E)-1-(2,6,6-trimethylcyclohex-2-en-1-yl) pent-1-en-3-one and mixtures thereof (e.g. ISORALDEINE), (E)-4-((3aS,7aS)-hexahydro-1H-4,7-methanoinden-5 (6H)-ylidene) butanal (e.g. DUPICAL), 3-(4-(2-methylpropyl)-2-methylphenyl) propanal (e.g. NYMPHEAL), 3-methyl-5-phenylpentan-1-ol (e.g. MEFROSOL), tetrahydro-4-methyl-2-(2-methylpropyl)-2H-pyran-4-ol (e.g. FLOROSA), 1-methyl-2-(5-methylhex-4-en-2-yl)cyclopropylmethanol (e.g. ROSYFOLIA); and
at least one sweet fragrance ingredient selected from benzo[d][1,3]dioxole-5-carbaldehyde (e.g. HELIOTROPINE), ethyl 2-(2-methyl-1,3-dioxolan-2-yl)acetate (e.g. FRUCTONE), 3-hydroxy-2-methyl-4H-pyran-4-one (e.g. MALTOL), and mixtures thereof.

In an embodiment, the scalp malodour counteracting fragrance composition of the present invention comprises:
at least 1 wt. % of 3,7-dimethyloct-6-en-1-ol (e.g. CITRONELLOL), 5-hexyloxolan-2-one (e.g. DECALACTONE GAMMA), or a mixture thereof, preferably a mixture thereof; and
at least 1, preferably at least 2, more preferably at least 3 fragrance ingredients selected from the group consisting of 1-phenylethanone (e.g. ACETOPHENONE); (3aR,5aS,9aS,9bR)-3a,6,6,9a-tetramethyl-2,4,5,5a,7,8,9,9b-octahydro-1H-benzo[e][1]benzo-furan (e.g. AMBROFIX, AMBROXAN or AMBROX); (E)-1-methoxy-4-(prop-1-en-1-yl)benzene (e.g. ANETHOLE); 3-(4-methoxyphenyl)-2-methylpropanal (e.g. FENNALDEHYDE); 5-tert-butyl-2-methyl-5-propyl-2H-furan (e.g. CASSYRANE); (E)-1-(2,6,6-trimethylcyclohexa-1,3-dien-1-yl) but-2-en-1-one (e.g. DAMASCENONE); (E)-1-(2,6,6-trimethylcyclohex-2-en-1-yl) but-2-en-1-one (e.g. DAMASCONE ALPHA); (E)-1-(2,6,6-trimethyl-1-cyclohexenyl) but-2-en-1-one (e.g. DAMASCONE BETA); (E)-2-methoxy-4-(prop-1-en-1-yl) phenol (e.g. ISOEUGENOL); 2-methoxy-4-propylphenol (e.g. DIHYDRO EUGENOL); 4-allyl-2-methoxyphenol (e.g. EUGENOL); (2R,8aS)-3′,6-dimethyl-3,4,4a,5,8,8a-hexahydro-1H-spiro[1,4-methano-naphthalene-2,2′-oxirane] (e.g. RHUBOFIX), anise and star anise oils, blackcurrant oils and reconstitutions, and mixtures thereof.

The malodour counteracting fragrance composition of the present invention may optionally comprise one or more additional fragrance ingredients not belonging to either one of Groups A, more particularly A1, A2 and A3, and B. In particular, the malodour counteracting fragrance composition of the present invention may comprise one or more fragrance ingredients selected from the group consisting of (2-(1-ethoxyethoxy)ethyl)benzene (e.g. ACETAL E); allyl 2-phenoxy-acetate (e.g. ACETATE PA); (E)-2-methoxy-4-(prop-1-en-1-yl)phenyl acetate (e.g. ACETYL ISOEUGENOL); 2,6,10-trimethylundec-9-enal (e.g. ADOXAL); dodecan-1-ol (e.g. ALCOHOL C 12 LAURIC); tridecan-1-ol (e.g. ALCOHOL C 13 OXO); hexan-1-ol (e.g. ALCOHOL C 6 HEXYLIC); decanal (e.g. ALDEHYDE C 10 DECYLIC); 2-methyldecanal (e.g. ALDEHYDE C 11 MOA); undec-10-enal (e.g. ALDEHYDE C 11 UNDECYLENIC); dodecanal (e.g. ALDEHYDE C 12 LAURIC); hexan-1-al (e.g. ALDEHYDE C 6 HEXYLIC); octanal (e.g. ALDEHYDE C 8 OCTYLIC); 3,5,5-trimethylhexanal (e.g. ALDEHYDE C 9 ISONONYLIC); nonanal (e.g. ALDEHYDE C 9 NONYLIC); (E)-dodec-2-enal (e.g. ALDEHYDE MANDARINE); 2,6-dimethylheptan-4-yl acetate (e.g. ALICATE); prop-2-enyl 2-(3-methylbutoxy)acetate (e.g. ALLYL AMYL GLYCOLATE); prop-2-enyl hexanoate (e.g. ALLYL CAPROATE); prop-2-enyl heptanoate (e.g. ALLYL OENANTHATE); decahydro-2,2,6,6,7,8,8-heptamethyl indenofuran (e.g. AMBER XTREME); 3,8,8,11a-tetramethyldodecahydro-1H-3,5a-epoxynaphtho[2,1-c] oxepine (e.g. AMBERKETAL); 2-(2,2,7,7-tetramethyltricyclo[6.2.1.0](1,6) undec-4-en-5-yl) propan-1-ol, 2-(2,2,7,7-tetramethyl-tricyclo[6.2.1.0] (1,6) undec-5-en-5-yl) propan-1-ol and mixtures thereof (e.g. AMBERMAX); (Z)-oxacycloheptadec-10-en-2-one (e.g. AMBRETTOLIDE); pentyl butanoate (e.g. AMYL BUTYRATE); 1-(3,3-dimethylcyclohexyl)ethyl formate (e.g. APHERMATE); (E)-methyl 2-((7-hydroxy-3,7-dimethyloctylidene)amino)benzoate (e.g. AURANTIOL PURE); (1R,2S,4R)-2′-isopropyl-1,7,7-trimethylspiro[bicyclo[2.2.1]heptane-2,4′-[1,3]dioxane] (e.g. BELAMBRE); benzaldehyde; benzyl alcohol, benzyl benzoate; benzyl 3-phenylprop-2-enoate (e.g. BENZYL CINNAMATE); benzyl 2-hydroxybenzoate (e.g. BENZYL SALICYLATE); octahydro-2H-chromen-2-one (e.g. BICYCLO NONALACTONE); (E)-1-methyl-4-(6-methylhepta-2,5-dien-2-yl)cyclohex-1-ene (e.g. BISABOLENE); (ethoxymethoxy)-cyclododecane (e.g. BOISAMBRENE FORTE); (1S,2S,4S)-1,7,7-trimethylbicyclo[2.2.1]heptan-2-ol (e.g. BORNEOL); (2S,4S)-1,7,7-trimethyl-bicyclo[2.2.1]heptan-2-yl acetate (e.g. BORNYL ACETATE); 3-(4-(tert-butyl)phenyl) propanal (e.g. BOURGEONAL); (1R,5S,E)-1,5-dimethylbicyclo[3.2.1]octan-8-one oxime (e.g. BUCCOXIME); butyl acetate; 4-(tert-butyl)cyclohexyl acetate (e.g. BUTYL CYCLOHEXYL ACETATE PARA); (4Z)-4, 11, 11-trimethyl-8-methylenebicyclo (7.2.0) undec-4-ene (e.g. CARYOPHYLLENE); 1,1,2,3,3-pentamethyl-2,3,6,7-tetrahydro-1H-inden-4 (5H)-one (e.g. CASHMERAN); (1R,6S,8aS)-6-methoxy-1,4,4,6-tetramethyloctahydro-1H-5,8a-methanoazulene (e.g. CEDRYL METHYL ETHER); 1,6-dioxacycloheptadecan-7-one (e.g. CERVOLIDE); (E)-3-phenylprop-2-en-1-ol (e.g. CINNAMIC ALCOHOL); (E)-3-phenylprop-2-en-1-yl acetate (e.g. CINNAMYL ACETATE); (E)-3,7-dimethylocta-2,6-dienal (e.g. CITRAL); 3,7-dimethyloct-6-en-1-yl formate (e.g. CITRONELLYL FORMATE); 3,7-dimethyloct-6-enenitrile (e.g. CITRONELLYL NITRILE); dodecanenitrile (e.g. CLONAL); (4S)-4,7,7-trimethyl-6-thiabicyclo[3.2.1]octane (e.g. CORPS PAMPLEMOUSSE); 2-hydroxy-3-methylcyclopent-2-enone (e.g. CORYLONE DRIED); (Z)-3-methylcyclotetradec-5-enone (e.g. COSMONE); 2H-chromen-2-one (e.g. COUMARIN); 2-methoxy-4-methylphenol (e.g. CREOSOL); p-cresol; 1-methoxy-4-methylbenzene (e.g. CRESYL METHYL ETHER PARA); ethyl 2,6,6-trimethylcyclohexa-1,3-diene-1-carboxylate (e.g. CRISTALON); 4-isopropylbenzonitrile (e.g. CUMIN NITRILE); 4-isopropylbenzaldehyde (e.g. CUMINIC ALDEHYDE); 3-(4-isopropylphenyl)-2-methylpropanal (e.g. CYCLAMEN ALDEHYDE); 8,8-dimethyl-1,2,3,4,5,6,7,8-octahydronaphthalene-2-carbaldehyde (e.g. CYCLOMYRAL); 1-methyl-4-propan-2-ylbenzene (e.g. CYMENE PARA); (E)-1-(2,6,6-trimethylcyclohexa-1,3-dien-1-yl) but-2-en-1-one (e.g. DAMASCENONE); 1-(2,6,6-trimethyl-1-cyclohex-3-enyl) but-2-en-1-one (e.g. DAMASCONE DELTA); 6-pentyltetrahydro-2H-pyran-2-one (e.g. DECALACTONE DELTA); (1S,6S)-3,7,7-trimethylbicyclo[4.1.0]hept-3-ene (e.g. DELTA-3 CARENE); diethyl propanedioate (e.g. DIETHYL MALONATE); dihexyl-but-2-enedioate (e.g. DIHEXYL FUMARATE); 3-methyl-2-pentylcyclopent-2-enone (e.g. DIHYDRO JASMONE); 2,6-dimethyloct-7-en-2-ol (e.g. DIHYDRO MYRCENOL); methyl 2-(methylamino)benzoate (e.g. DIMETHYL ANTHRANILATE); 2-methyl-4-phenylbutan-2-ol (e.g. DIMETHYL PHENYL ETHYL CARBINOL); (methylsulfanyl) methane (e.g. DIMETHYL SULPHIDE); 2,6-dimethylheptan-2-ol (e.g. DIMETOL); oxydibenzene (e.g. DIPHENYL OXIDE); (E)-dodec-2-enal (e.g. DODECENAL); 1-methoxy-7,9-dimethyl-2-oxaspiro[5.5]undec-8-ene (e.g. EBELIA); ethyl acetate (e.g. ETHYL ACETATE); ethyl 3-oxobutanoate (e.g. ETHYL ACETOACETATE); ethyl benzoate (e.g. ETHYL BENZOATE); ethyl butanoate (e.g. ETHYL BUTYRATE); ethyl hexanoate (e.g. ETHYL HEXANOATE); 6-methylheptan-3-one (e.g. ETHYL ISOAMYL KETONE); (E)-3,7-dimethylnona-1,6-dien-3-ol (e.g. ETHYL LINALOOL); 2-ethyl-3-hydroxy-4H-pyran-4-one (e.g. ETHYL MALTOL); ethyl 2-methylbutanoate (e.g. ETHYL METHYL-2-BUTYRATE); ethyl 3-phenyloxirane-2-carboxylate (e.g. ETHYL PHENYL GLYCIDATE); ethyl propanoate (e.g. ETHYL PROPIONATE); 3-ethoxy-4-hydroxybenzaldehyde (e.g. ETHYL VANILLIN); 1,4-dioxacycloheptadecane-5,17-dione (e.g. ETHYLENE BRASSYLATE); (1S,4S)-1,3,3-trimethyl-2-oxabicyclo[2.2.2]octane (e.g. EUCALYPTOL); methyl 2,4-dihydroxy-3,6-di-methylbenzoate (e.g. EVERNYL); 3-(4-ethylphenyl)-2,2-dimethylpropanal (e.g. FLORALOZONE); 3-(3-isopropylphenyl) butanal (e.g. FLORHYDRAL); (E)-undec-9-enenitrile (e.g. FLORIDILE); 2-(sec-butyl)cyclohexanone (e.g. FRESKOMENTHE); (3aS,4S,7R,7aS)-ethyl octahydro-1H-4,7-methanoindene-3a-carboxylate (e.g. FRUITATE); 1-(5,5-dimethylcyclohex-1-en-1-yl) pent-4-en-1-one (e.g. GALBANONE); (3aR,6S,7aS)-3a,4,5,6,7,7a-hexahydro-1H-4,7-methanoinden-6-yl 2-methyl propanoate (e.g. GARDOCYCLENE); (E)-3,7-dimethylocta-2,6-dien-1-ol (e.g. GERANIOL 980); (E)-3,7-dimethylocta-2,6-dien-1-ol (e.g. GERANIOL); (E)-3,7-dimethylocta-2,6-dien-1-yl acetate (e.g. GERANYL ACETATE); (E)-3,7-dimethylocta-2,6-dien-1-yl butanoate (e.g. GERANYL BUTYRATE); (E)-3,7-dimethylocta-2,6-dien-1-yl formate (e.g. GERANYL FORMATE); (E)-(E)-3,7-dimethylocta-2,6-dien-1-yl 2-methylbut-2-enoate (e.g. GERANYL TIGLATE); ethyl 2-ethyl-6,6-dimethylcyclohex-2-enecarboxylate (e.g. GIVESCONE); (E)-oxacyclohexadec-12-en-2-one (e.g. HABANOLIDE); methyl 3-oxo-2-pentylcyclopentaneacetate (e.g. HEDIONE); [2-[1-(3,3-dimethylcyclohexyl) ethoxy]-2-methylpropyl]propanoate (e.g. HELVETOLIDE); 5-propyloxolan-2-one (e.g. HEPTALACTONE GAMMA); methyl 7-isopropyl-1,4a-dimethyl-1,2,3,4,4a,4b,5,6,7,8,10, 10a-dodecahydrophenanthrene-1-carboxylate (e.g. HERCOLYN DW); (E)-hex-2-enal (e.g. HEXENAL-2-TRANS); (E)-hex-2-en-1-ol (e.g. HEXENOL-2-TRANS); (Z)-hex-3-en-1-ol (e.g. HEXENOL-3-CIS); (E)-hex-3-en-1-yl acetate (e.g. HEXENYL ACETATE TRANS); (Z)-hex-3-en-1-yl acetate (e.g. HEXENYL-3-CIS ACETATE); (Z)-hex-3-en-1-yl butanoate (e.g. HEXENYL-3-CIS BUTYRATE); (Z)-hex-3-en-1-yl 2-methylpropanoate (e.g. HEXENYL-3-CIS ISOBUTYRATE); (Z)-hex-3-en-1-yl propanoate (e.g. HEXENYL-3-CIS PROPIONATE); (Z)-hex-3-en-1-yl 2-hydroxybenzoate (e.g. HEXENYL-3-CIS SALICYLATE); hexyl acetate (e.g. HEXYL ACETATE); hexyl butanoate (e.g. HEXYL BUTYRATE); hexyl hexanoate (e.g. HEXYL HEXANOATE); hexyl 2-methylpropanoate (e.g. HEXYL ISOBUTYRATE); hexyl propanoate (e.g. HEXYL PROPIONATE); hexyl 2-hydroxybenzoate (e.g. HEXYL SALICYLATE); (1,1-dimethoxypropan-2-yl)benzene (e.g. HYDRATROPIC ALDEHYDE DIMETHYL ACETAL); 4-ethyl-5-methylthiazol-2-ol (e.g. HYDROXY ETHYL METHYL THIAZOL); 4,4a,5,9b-tetrahydroindeno[1,2-d][1,3]dioxine (e.g. INDOFLOR); 3-methylbutyl acetate (e.g. ISOAMYL ACETATE); 3-methylbutyl butanoate (e.g. ISOAMYL BUTYRATE FR); 2-methylpropyl 2-methylpropanoate (e.g. ISOBUTYL ISOBUTYRATE); 2-isopropyl-5-methylcyclohexanone (e.g. ISOMENTHONE DL); 3,5,5-trimethylhexyl acetate (e.g. ISONONANYL ACETATE); 3-methylbutyl 3-methylbutanoate (e.g. ISOPENTYL ISOVALERATE); isopropyl 2-methylbutanoate (e.g. ISOPROPYL METHYL-2-BUTYRATE); 2-isopropyl-4-methylthiazole (e.g. ISOPROPYL-2 METHYL-4 THIAZOLE); (E)-6-(pent-3-en-1-yl)tetrahydro-2H-pyran-2-one (e.g. JASMOLACTONE); 2-methyl-3-(4-methylphenyl) propanal (e.g. JASMORANGE); (4Z)-hept-4-en-2-yl 2-hydroxybenzoate (e.g. KARMAFLOR); (Z)-3,4,5,6,6-pentamethylhept-3-en-2-one (e.g. KOAVONE); (3E,6E)-2,4,4,7-tetramethylnona-6,8-dien-3-one oxime (e.g. LABIENOXIME); 2,2,6-trimethyl-6-vinyltetrahydro-2H-pyran (e.g. LIMETOL); 2-(5-methyl-5-vinyltetrahydrofuran-2-yl) propan-2-ol (e.g. LINALOOL OXIDE); 3,7-dimethylocta-1,6-dien-3-ol (e.g. LINALOOL); 3,7-dimethylocta-1,6-dien-3-yl acetate (e.g. LINALYL ACETATE); 3,7-dimethylocta-1,6-dien-3-yl 3-phenylprop-2-enoate (e.g. LINALYL CINNAMATE); 3,7-dimethylocta-1,6-dien-3-yl formate (e.g. LINALYL FORMATE); 3,7-dimethylocta-1,6-dien-3-yl 2-methylpropanoate (e.g. LINALYL ISOBUTYRATE); bicyclo[2.2.2]oct-5-ene-2-carboxaldehyde (e.g. MACEAL); 2,4-dimethyl-4,4a,5,9b-tetrahydroindeno[1,2-d][1,3]dioxine (e.g. MAGNOLAN); 2,2-dimethyl-3-(m-tolyl) propan-1-ol (e.g. MAJANTOL); 2-methyl-4-oxo-4H-pyran-3-yl 2-methylpropanoate (e.g. MALTYL ISOBUTYRATE); ethyl 2-methylpentanoate (e.g. MANZANATE); 3-methyl-5-phenylpentanal (e.g. MEFRANAL); 2,6-dimethylhept-5-enal (e.g. MELONAL); 2-isopropyl-5-methylcyclohexanol (e.g. MENTHOL); 2-isopropyl-5-methylcyclohexanone (e.g. MENTHONE); 2-methoxy-3-methylpyrazine (e.g. METHOXY METHYL PYRAZINE); 4-(4-methoxyphenyl) butan-2-one (e.g. METHOXY PHENYL BUTANONE); methyl benzoate (e.g. METHYL BENZOATE); methyl 3-phenylprop-2-enoate (e.g. METHYL CINNAMATE); 5-hexyl-5-methyloxolan-2-one (e.g. METHYL DECALACTONE GAMMA); 2-ethoxy-4-(methoxymethyl) phenol (e.g. METHYL DIANTILIS); 6-methylhept-5-en-2-one (e.g. METHYL HEPTENONE); methyl 2-methylbutanoate (e.g. METHYL METHYL-2 BUTYRATE); 6,6-dimethoxy-2,5,5-trimethylhex-2-ene (e.g. METHYL PAMPLEMOUSSE); methyl 2-hydroxybenzoate (e.g. METHYL SALICYLATE); 2-methyl pyrazine (e.g. METHYL-2 PYRAZINE); 1a,3,3,4,6,6-hexamethyl-1a,2,3,4,5,6,7,7a-octahydronaphtho[2,3-b] oxirene (e.g. MOXALONE); 1,4-dioxacyclohexadecane-5,16-dione (e.g. MUSK C14); (4-(4-methylpent-3-en-1-yl)cyclohex-3-en-1-yl)methyl acetate (e.g. MYRALDYL ACETATE); ethyl 2-ethyl-3,6,6-trimethylcyclohexene-1-carboxylate (e.g. MYRASCONE); 2-(2-(4-methylcyclohex-3-en-1-yl) propyl)cyclopentan-1-one (e.g. NECTARYL); 10-isopropyl-2,7-dimethyl-1-oxaspiro[4.5]deca-3,6-diene (e.g. NEOCASPIRENE EXTRA); (2E,6Z)-nona-2,6-dienal (e.g. NONADIENAL); (2Z,6E)-2,6-nonadien-1-ol (e.g. NONADIENOL-2,6); (Z)-non-6-en-1-ol (e.g. NONENOL-6-CIS); (E)-3,7-dimethylocta-1,3,6-triene (e.g. OCIMENE); 2,4-dimethyl-2-(5,5,8,8-tetramethyl-5,6,7,8-tetrahydronaphthalen-2-yl)-1,3-dioxolane (e.g. OKOUMAL); 7-methoxy-3,7-dimethyloctan-2-ol (e.g. OSYROL); 2,4a,5,8a-tetramethyl-1,2,3,4,4a,7,8,8a-octahydronaphthalen-1-yl formate (e.g. OXYOCTALINE FORMATE); 2-ethyl-N-methyl-N-(m-tolyl) butanamide (e.g. PARADISAMIDE); 5-heptyldihydrofuran-2 (3H)-one (e.g. PEACH PURE); 2-cyclohexylidene-2-(o-tolyl) acetonitrile (e.g. PETALIA); 2-cyclohexylhepta-1,6-dien-3-one (e.g. PHARAONE); 2-phenoxyethanol (e.g. PHENOXY ETHYL ALCOHOL); 2-phenylethyl formate (e.g. PHENYL ETHYL FORMATE); 2-phenylethyl 2-methylpropanoate (e.g. PHENYL ETHYL ISOBUTYRATE); 3-phenylpropan-1-ol (e.g. PHENYL PROPYL ALCOHOL); 2,6,6-trimethylbicyclo[3.1.1]hept-2-ene (e.g. PINENE ALPHA); 6,6-dimethyl-2-methylenebicyclo[3.1.1]heptane (e.g. PINENE BETA); 2,2-dimethyl-2-pheylethyl propanoate (e.g. PIVAROSE); (4aS,8aR)-7-methyloctahydro-1,4-methanonaphthalen-6 (2H)-one (e.g. PLICATONE); 1-methyl-4-(4-methylpent-3-en-1-yl)cyclohex-3-enecarbaldehyde (e.g. PRECYCLEMONE B); 3-methylbut-2-en-1-yl acetate (e.g. PRENYL ACETATE); 5-pentyldihydrofuran-2 (3H)-one (e.g. PRUNOLIDE); (E)-2-ethyl-4-(2,2,3-trimethylcyclopent-3-en-1-yl) but-2-en-1-ol (e.g. RADJANOL); 4-(4-hydroxyphenyl) butan-2-one (e.g. RASPBERRY KETONE); 2,2,2-trichloro-1-phenylethyl acetate (e.g. ROSACETOL); 4-methyl-2-(2-methylprop-1-en-1-yl)tetrahydro-2H-pyran (e.g. ROSE OXIDE); 1,1-diethoxycyclohexane (e.g. RUM ACETAL); 3-methyl-5-(2,2,3-trimethylcyclopent-3-en-1-yl) pentan-2-ol (e.g. SANDALORE); 3-((1R,2S,4R, 6R)-5,5,6-trimethylbicyclo[2.2.1]heptan-2-yl)cyclohexanol (e.g. SANDELA); ethyl N,S-bis(4-oxo-4-(2,6,6-trimethylcyclohex-3-en-1-yl) butan-2-yl) cysteinate (e.g. SCENTAURUS BERRY); 4-(dodecylthio)-4-methylpentan-2-one (e.g. SCENTAURUS JUICY); (3aR,4R,6S,7R,7aR)-6-methoxyoctahydro-1H-4,7-methanoindene-1-carbaldehyde (e.g. SCENTENAL); 2-(1-(3,3-dimethylcyclohexyl) ethoxy)-2-methylpropyl cyclopropanecarboxylate (e.g. SERENOLIDE); 2-methyl-3-[4-(2-methylpropyl)phenyl] propanal (e.g. SILVIAL); 1-(spiro[4.5]dec-6-en-7-yl) pent-4-en-1-one (e.g. SPIROGALBANONE); 1-phenylethyl acetate (e.g. STYRALLYL ACETATE); 1-methyl-4-propan-2-ylcyclohexa-1,4-diene (e.g. TERPINENE GAMMA); 2-(4-methyl-1-cyclohex-3-enyl) propan-2-ol (e.g. TERPINEOL ALPHA); 1-methyl-4-(propan-2-ylidene)cyclohex-1-ene (e.g. TERPINOLENE); 2-(4-methyl-1-cyclohex-3-enyl) propan-2-yl acetate (e.g. TERPINYL ACETATE); 3,7-dimethyloctan-3-yl acetate (e.g. TETRAHYDRO LINALYL ACETATE); oxacyclohexadecan-2-one (e.g. THIBETOLIDE); 2-isopropyl-5-methylphenol (e.g. THYMOL CRYSTALS); triethyl 2-hydroxypropane-1,2,3-tricarboxylate (e.g. TRIETHYL CITRATE); 3-(benzo[d][1,3]dioxol-5-yl)-2-methylpropanal (e.g. TROPIONAL); (3E,5Z)-undeca-1,3,5-triene (e.g. UNDECATRIENE); (E)-4-methyldec-3-en-5-ol (e.g. UNDECAVERTOL); 5-methyloxolan-2-one (e.g. VALEROLACTONE GAMMA); 4-hydroxy-3-methoxybenzaldehyde (e.g. VANILLIN); 3,4-dimethoxybenzaldehyde (e.g. VERATRYL ALDEHYDE); (E)-methyl 2-((3-(4-(tert-butyl)phenyl)-2-methylprop-1-en-1-yl)amino)benzoate (e.g. VERDANTIOL); 1-methyl-4-(4-methylpentyl)cyclohex-3-enecarbaldehyde (e.g. VERNALDEHYDE); 4-methyl-4-phenylpentan-2-yl acetate (e.g. VETIKOL ACETATE/CORPS RHUBARB); (2E,6Z)-nona-2,6-dienenitrile (e.g. VIOLET NITRILE); undec-10-enenitrile (e.g. VIOLIFF); (2,2-dimethoxyethyl)benzene (e.g. VIRIDINE); bergamot oils, cassia oils, eucalyptus oils, ginger oils, grapefruit oils and terpenes, jasmine oils, lemon oils, lime oils, lemon terpenes, grapefruit terpenes, mandarin oils, peppermint oils, sandalwood oils, tuberose oils and mixtures thereof.

Certain of these additional fragrance ingredients have been found to enhanced the scalp malodour counteractancy of the fragrance composition according to the present invention and are therefore preferred.

In an embodiment, the scalp malodour counteracting fragrance composition additionally comprises from 15 to 60 wt. %, more particularly 20 to 50 wt. % of at least 1, preferably at least 2, preferably at least 3 fragrance ingredients drawn from the group consisting of (E)-2-methoxy-4-(prop-1-en-1-yl)phenyl acetate (e.g. ACETYL ISOEUGENOL); 2,6,10-trimethylundec-9-enal (e.g. ADOXAL); tridecan-1-ol (e.g. ALCOHOL C 13 OXO); decanal (e.g. ALDEHYDE C 10 DECYLIC); 2-methyldecanal (e.g. ALDEHYDE C 11 MOA); undec-10-enal (e.g. ALDEHYDE C 11 UNDECYLENIC); dodecanal (e.g. ALDEHYDE C 12 LAURIC); hexan-1-al (e.g. ALDEHYDE C 6 HEXYLIC); octanal (e.g. ALDEHYDE C 8 OCTYLIC); 3,5,5-trimethylhexanal (e.g. ALDEHYDE C 9 ISONONYLIC); nonanal (e.g. ALDEHYDE C 9 NONYLIC); (E)-dodec-2-enal (e.g. ALDEHYDE MANDARINE); prop-2-enyl 2-(3-methylbutoxy)acetate (e.g. ALLYL AMYL GLYCOLATE); prop-2-enyl hexanoate (e.g. ALLYL CAPROATE); prop-2-enyl heptanoate (e.g. ALLYL OENANTHATE); decahydro-2,2,6,6,7,8,8-heptamethyl indenofuran (e.g. AMBER XTREME); 3,8,8,11a-tetramethyldodecahydro-1H-3,5a-epoxynaphtho[2,1-c] oxepine (e.g. AMBERKETAL); 2-(2,2,7,7-tetramethyltricyclo[6.2.1.0] (1,6) undec-4-en-5-yl) propan-1-ol and 2-(2,2,7,7-tetramethyltricyclo [6.2.1.0] (1,6) undec-5-en-5-yl) propan-1-ol (e.g. AMBERMAX); (E)-methyl 2-((7-hydroxy-3,7-dimethyloctylidene)amino)benzoate (e.g. AURANTIOL PURE); benzyl 3-phenylprop-2-enoate (e.g. BENZYL CINNAMATE); benzyl 2-hydroxybenzoate (e.g. BENZYL SALICYLATE); octahydro-2H-chromen-2-one (e.g. BICYCLO NONALACTONE); (1S,2S,4S)-1,7,7-trimethylbicyclo[2.2.1]heptan-2-ol (e.g. BORNEOL); 3-(4-(tert-butyl)phenyl) propanal (e.g. BOURGEONAL); 4-(tert-butyl)cyclohexyl acetate (e.g. BUTYL CYCLOHEXYL ACETATE PARA); 1,1,2,3,3-penta-methyl-2,3,6,7-tetrahydro-1H-inden-4 (5H)-one (e.g. CASHMERAN); (E)-3-phenylprop-2-en-1-ol (e.g. CINNAMIC ALCOHOL); (E)-3,7-dimethylocta-2,6-dienal (e.g. CITRAL); 3,7-dimethyloct-6-enenitrile (e.g. CITRONELLYL NITRILE); 2-hydroxy-3-methylcyclopent-2-enone (e.g. CORYLONE); 2H-chromen-2-one (e.g. COUMARIN PURE); 4-isopropylbenzaldehyde (e.g. CUMINIC ALDEHYDE); 3-(4-isopropylphenyl)-2-methylpropanal (e.g. CYCLAMEN ALDEHYDE EXTRA); 8,8-dimethyl-1,2,3,4,5,6,7,8-octahydronaphthalene-2-carbaldehyde (e.g. CYCLO-MYRAL); (E)-1-(2,6,6-trimethylcyclohexa-1,3-dien-1-yl) but-2-en-1-one (e.g. DAMASCENONE); 1-(2,6,6-trimethyl-1-cyclohex-3-enyl) but-2-en-1-one (e.g. DAMASCONE DELTA); 2,6-dimethyloct-7-en-2-ol (e.g. DIHYDRO MYRCENOL); 2,6-dimethylheptan-2-ol (e.g. DIMETOL); (E)-dodec-2-enal (e.g. DODECENAL); ethyl benzoate (e.g. ETHYL BENZOATE); (E)-3,7-dimethylnona-1,6-dien-3-ol (e.g. ETHYL LINALOOL); 2-ethyl-3-hydroxy-4H-pyran-4-one (e.g. ETHYL MALTOL); ethyl 2-methylbutanoate (e.g. ETHYL METHYL-2-BUTYRATE); 1,4-dioxacycloheptadecane-5,17-dione (e.g. ETHYLENE BRASSYLATE); (1S,4S)-1,3,3-trimethyl-2-oxabicyclo[2.2.2]octane (e.g. EUCALYPTOL); 3-(4-ethylphenyl)-2,2-dimethylpropanal (e.g. FLORALOZONE); 3-(3-isopropylphenyl) butanal (e.g. FLORHYDRAL); 1-(5,5-dimethylcyclohex-1-en-1-yl) pent-4-en-1-one (e.g. GALBANONE PURE); (E)-3,7-dimethylocta-2,6-dien-1-ol (e.g. GERANIOL 980); (E)-3,7-dimethylocta-2,6-dien-1-yl acetate (e.g. GERANYL ACETATE); (E)-3,7-dimethylocta-2,6-dien-1-yl formate (e.g. GERANYL FORMATE); (E)-hex-2-enal (e.g. HEXENAL-2-TRANS); (E)-hex-2-en-1-ol (e.g. HEXENOL-2-TRANS); (Z)-hex-3-en-1-ol (e.g. HEXENOL-3-CIS); (Z)-hex-3-en-1-yl acetate (e.g. HEXENYL-3-CIS ACETATE); (Z)-hex-3-en-1-yl 2-hydroxybenzoate (e.g. HEXENYL-3-CIS SALICYLATE); hexyl acetate; (1,1-dimethoxypropan-2-yl)benzene (e.g. HYDRATROPIC ALDEHYDE DIMETHYL ACETAL); (4Z)-hept-4-en-2-yl 2-hydroxybenzoate (e.g. KARMAFLOR); (Z)-hex-3-en-1-yl methyl carbonate (e.g. LIFFAROME); 2,2,6-trimethyl-6-vinyltetrahydro-2H-pyran (e.g. LIMETOL); 3,7-dimethylocta-1,6-dien-3-ol (e.g. LINALOOL); 3,7-dimethylocta-1,6-dien-3-yl acetate (e.g. LINALYL ACETATE); 3,7-dimethylocta-1,6-dien-3-yl 2-methylpropanoate (e.g. LINALYL ISOBUTYRATE); bicyclo[2.2.2]oct-5-ene-2-carboxaldehyde (e.g. MACEAL); 2,2-dimethyl-3-(m-tolyl) propan-1-ol (e.g. MAJANTOL); ethyl 2-methylpentanoate (e.g. MANZANATE); 3-methyl-5-phenylpentanal (e.g. MEFRANAL); 2,6-dimethylhept-5-enal (e.g. MELONAL); methyl benzoate (e.g. METHYL BENZOATE); methyl 2-methylbutanoate (e.g. METHYL METHYL-2 BUTYRATE); (2E,6Z)-nona-2,6-dienal (e.g. NONADIENAL); (2Z,6E)-2,6-nonadien-1-ol (e.g. NONADIENOL-2,6); (Z)-non-6-en-1-ol (e.g. NONENOL-6-CIS); 2-cyclohexylhepta-1,6-dien-3-one (e.g. PHARAONE); 2-phenoxyethanol (e.g. PHENOXY ETHYL ALCOHOL); 3-phenylpropan-1-ol (e.g. PHENYL PROPYL ALCOHOL); 3-methylbut-2-en-1-yl acetate (e.g. PRENYL ACETATE); 5-pentyldihydrofuran-2 (3H)-one (e.g. PRUNOLIDE); (E)-2-ethyl-4-(2,2,3-trimethylcyclopent-3-en-1-yl) but-2-en-1-ol (e.g. RADJANOL); 4-(4-hydroxyphenyl) butan-2-one (e.g. RASPBERRY KETONE); 4-methyl-2-(2-methylprop-1-en-1-yl)tetrahydro-2H-pyran (e.g. ROSE OXIDE); (3aR,4R,6S,7R,7aR)-6-methoxyoctahydro-1H-4,7-methanoindene-1-carbaldehyde (e.g. SCENTENAL); 2-methyl-3-[4-(2-methylpropyl)phenyl] propanal (e.g. SILVIAL); 1-(spiro[4.5]dec-6-en-7-yl) pent-4-en-1-one (e.g. SPIROGALBANONE); 1-phenylethyl acetate (e.g. STYRALLYL ACETATE); 2-(4-methyl-1-cyclohex-3-enyl) propan-2-ol (e.g. TERPINEOL ALPHA); 2-(4-methyl-1-cyclohex-3-enyl) propan-2-yl acetate (e.g. TERPINYL ACETATE); oxacyclohexadecan-2-one (e.g. THIBETOLIDE); 2-isopropyl-5-methylphenol (e.g. THYMOL CRYSTALS); 3-(benzo[d][1,3]dioxol-5-yl)-2-methylpropanal (e.g. TROPIONAL); (E)-4-methyldec-3-en-5-ol (e.g. UNDECAVERTOL); 3,4-dimethoxybenzaldehyde (e.g. VERATRYL ALDEHYDE); (E)-methyl 2-((3-(4-(tert-butyl)phenyl)-2-methylprop-1-en-1-yl)amino)benzoate (e.g. VERDANTIOL); 1-methyl-4-(4-methylpentyl)cyclohex-3-enecarbaldehyde (e.g. VERNALDEHYDE); (2E,6Z)-nona-2,6-dienenitrile (e.g. VIOLET NITRILE); and mixtures thereof.

As mentioned hereinabove, the scalp malodour counteracting fragrance composition may comprise solvents in addition to the fragrance ingredients mentioned hereinabove.

In an embodiment, the scalp malodour counteracting fragrance composition further comprises from 0.1 wt. % to 50 wt. %, preferably from 0.5 wt. % to 40 wt. %, more preferably from 1 wt. % to 30 wt. % of a solvent selected from the group consisting of propylene glycol, dipropylene glycol, triethyl citrate, diethyl citrate, isopropylidene glycerol, 1,2-propane diol, 1,3-propane diol, 1,2-butane diol, 1,3-butane diol, 1,2-pentane diol, triacetine, dimethyl isosorbide, dimethyl carbonate, isoparaffinic hydrocarbons, and mixtures thereof.

The scalp malodour counteracting fragrance composition may optionally be encapsulated.

In one embodiment, the scalp malodour counteracting fragrance composition according to the present invention is emulsified in an aqueous phase comprising hydrophilic encapsulating materials and the emulsion is dried, typically by spray drying. Typical hydrophilic encapsulating materials include carbohydrates, such as maltodextrins and sugar alcohols. Typical emulsifiers include modified starches and gums, such as acacia gum. Such encapsulation methods and products are well known to the art. The resulting form is typically a hydrophilic powder or a hydrophilic granulate that releases the fragrance composition in contact with moisture.

Alternatively, in an embodiment, the scalp malodour counteracting fragrance composition according to the present invention is encapsulated in a core-shell microcapsule. A core-shell microcapsule comprises a core comprising the fragrance composition and a shell comprising an encapsulating material. In this case, the encapsulating material is typically a highly cross-linked material, such as a thermoset resin or a polymer network, the role of which is to prevent or mitigate the leakage of the fragrance composition out of the microcapsule core. Such encapsulation methods and products are also well known to the art. The resulting product is typically a slurry comprising a multitude of such microcapsules dispersed in an aqueous phase. The fragrance composition may be released by mechanical rupture of the shell or by slow diffusion through the shell.

In an embodiment, the shell of the core-shell microcapsule comprises a coacervate, preferably a complex coacervate involving two biopolymers, as described in co-pending patent application WO 2023/020883, which is herewith incorporated by reference.

In an embodiment, the slurry of core-shell microcapsules is dried to form a powder or a granulate.

In an embodiment, hydrophilic encapsulating materials are added to the aqueous phase of the slurry before drying.

In an embodiment, the hydrophilic powder, the dried core-shell microcapsules, or mixtures thereof are admixed, optionally with a carrier material, such as a salt, a clay, a polymer, a biopolymer, or a carbohydrate.

The present invention further provides a method of assessing the scalp malodour counteraction capability of a test fragrance composition, the method comprising the steps of:
a) Applying a product composition comprising the test fragrance composition onto a clean substrate;
b) Removing the shampoo composition by rinsing the substrate with water;
c) Letting the substrate dry in air;
d) Placing the substrate into a confined space, in the presence of a scalp malodour;
e) Closing the confined space;
f) Opening the confined space after equilibration and assessing both malodour intensity and fragrance intensity in this confined space;
wherein a scalp malodour counteraction capability is confirmed if the fragrance intensity is stronger than the malodour intensity.

In one particular embodiment of the method of the invention, the substrate is selected from materials that have features similar to human skin, including pig skin, artificial skin, and polyester fibres. Fabrics comprising or consisting of polyester are particularly well suited. The advantage of using a fabric is that it mimics better the scalp environment in terms of available surface area.

The dimension of the substrate may be chosen such that application and assessment are facilitated, as described hereinafter.

The substrate is wetted by dipping it once in cold water. The product composition comprising the fragrance composition to be tested is applied homogeneously onto this wet substrate by using a suitable means, for example a finger. The penetration of the product into the substrate may be achieved by folding the substrate in half and rubbing and massaging these two parts against each other for 30 seconds. The amount of product composition may be from 0.15 to about 0.6 g/m², for example about 0.3 g/m².

The level of fragrance composition in the product composition may be from about 0.5 to about 1.5 wt. %, typically about 1 wt. %

Once the product composition has been applied as described hereinabove, the substrate is rinsed with cold water. The rinse procedure preferably mimics the way a human scalp is exposed to rinse water in reality. A suitable procedure is to dip the substrate into cold water and pull it out immediately after it was entirely immersed. This gesture is preferably repeated five times in order to obtain a reproducible rinse.

The substrate may be dried under any suitable conditions, for example on a metal grid in contact with the ambient air and at room temperature. Drying time should be chosen such that the substrate is more or less dry, e.g. for a period of time from about 60 to about 240 minutes, more preferably from about 90 to 180 minutes, for example 120 minutes.

The confined space may be any closed environment. The advantage of having a confined space is that such a space allows the formation of an equilibrated headspace where both malodour ingredients and fragrance ingredients coexist and where the malodour counteracting capability of the fragrance composition can be optimally assessed. Any confined container may be used as confined space, including a booth, a box, a plastic bag of a glass jar, provided the container is odourless. For example, a jar may be used.

The container may typically be maintained at room temperature, for example at about 22±3° C.

The malodour may be provided in any suitable form in the container, e.g. as a liquid (solution) in a vessel or on a carrier, or sprayed onto the substrate mentioned hereinabove. For example, the malodour may be applied pure on a filter paper or cotton pad, or in the form of a solution in a suitable solvent, such as propylene glycol, dipropylene glycol, 1,2-butanediol, isopropylidene glycerol or a mixture thereof. The solution may contain from about 0.01 to 25 wt. %, typically from about 5 to about 15 wt. % of malodour, for example 10 wt. %.

The dimensions of the substrate and, consequently, the volume of the product applied to this substrate and the amount of malodour may have to be adapted to each other, so that the intensities of fragrance, malodour or mixtures thereof are perceivable but not overwhelming in the confined space. A suitable set-up may combine a jar having a volume of from about 100 to about 200 ml, for example about 125 ml. The dimensions of the substrate may be from about 20 to about 50 cm², typically from about 30 to about 40 cm², for example about 35 cm². The amount of applied product may be from about 0.05 to about 2 g, for example about 0.1 g. A suitable amount of malodour provided into the container may be from about 0.05 to about 2 g, for example about 0.1 g of a 10 wt. % solution.

In an alternative embodiment of the method according to the invention, the substrate is one or more hair swatches. The weight of the substrate may be from about 1 to about 30 g, typically from about 10 to about 20 g, for example about 15 g.

The amount of product per hair swatch may be from about 10 to about 20 wt. %, for example about 16 wt. %, based on the total weight of the neat swatches; the swatches may be rinsed with medium hot water having a temperature of 35+/−5° C. and the drying time may be from about 18 hours to about 30 hours, for example about 24 hours.

The malodour may be applied on a filter paper or cotton pad in the form of a solution in a suitable solvent, such as propylene glycol, dipropylene glycol, 1,2-butanediol, isopropylidene glycerol or a mixture thereof. The solution may contain from about 0.01 to about 20 wt. %, typically from about 0.1 to about 10 wt. % of malodour, for example about 1 wt. %.

A suitable set-up may combine a jar having a volume of from about 250 ml to about 1000 ml, for example about 500 ml. A suitable amount of malodour provided into the container may be from about 1 to about 10 μL, for example about 5 μL of a 1 wt. % solution.

In respect to step f), the equilibration time may be from about 15 minutes to about 60 minutes, for example about 30 minutes.

The fragrance intensity may be assessed by smelling by placing the nose directly at the opening of the jar.

Both malodour and fragrance intensity may be assessed on a scale of 1-5 (1=barely noticeable, 2=weak, 3=medium, 4=strong and 5=very strong).

An overall scalp malodour counteraction (SMOC) score of 0 (poor) maybe given if the malodour intensity is significantly higher than the fragrance intensity. An overall score of 1 (average) is given if the malodour intensity is similar to that of the fragrance intensity and a score of 2 (good) is given if the malodour intensity is significantly lower than the fragrance intensity. A difference of 1 score units is typically judged as significant.

The present invention also provides a cosmetic product, in particular a skin care or hair care product, comprising the scalp malodour counteracting fragrance composition of the present invention and a suitable carrier.

Skin care products include, but are not limited to, liquid and solid soaps, shower gels and creams.

Hair care products include, but are not limited to liquid or solid soaps, shower gels or creams, shampoos, leave-on or rinse-off hair conditioners, combing creams, solid shampoos, hair sprays, hair waxes, styling gels or styling creams, for instance.

Depending on the intended application, the carrier may be a solvent, base or solid support, for instance.

The malodour counteracting fragrance composition of the present invention is particularly suitable for masking, reducing and/or preventing scalp malodour.

Therefore, in an embodiment, the cosmetic product of the present invention is a product for application to the scalp, in particular during hair scalp wash.

In this case, the carrier used in the consumer product should be suitable for application to the human body, i.e. cosmetically acceptable. Any carriers commonly used in the preparation of cosmetic preparations for use on the human skin or hair may be employed in the present invention. Suitable carriers include, but are not limited to, liquids, such as water, oils or surfactants, including those of petroleum, animal, plant or synthetic origin, such as and not restricted to, peanut oil, soybean oil, mineral oil, sesame oil, castor oil, polysorbates, sorbitan esters, ether sulfates, sulfates, betaines, glycosides, maltosides, fatty alcohols, nonoxynols, poloxamers, polyoxyethylenes, polyethylene glycols, dextrose, glycerol, digitonin, and the like.

The consumer product may be produced in any solid, liquid, or semi-solid form useful for application to the skin. Thus, it may be a cream, an emulsion, a solution, an anhydrous composition, an aqueous dispersion, an oil, a milk, a balsam, a foam, an aqueous or oily lotion, an aqueous or oily gel, a slurry, an encapsulate, a hydro-alcoholic solution, a hydro-glycolic solution, a hydrogel, a liniment, a serum, a soap, an ointment, a pomade, a paste, a powder, a bar, a stick, flakes, beads, pellets, a spray or an aerosol (spray), including leave-on and rinse-off formulations.

Depending on the application, the cosmetic product of the present invention should contain more or less of the malodour counteracting fragrance composition of the present invention.

In an embodiment, the concentration of the scalp malodour counteracting fragrance composition in the cosmetic product may be from about 0.1 wt. % to about 5 wt. %, preferably from about 0.2 to about 2 wt. %, more preferably from about 0.35 wt. % to about 1.4 wt. %, based on the total weight of the cosmetic product, for example from about 0.5 to about 1.4 wt. % in a shampoo or from about 0.35 wt. % to about 1 wt. % in a hair conditioner.

The cosmetic product of the present invention may further comprise one or more additives, such as other actives and/or adjuvants.

In an embodiment, the cosmetic product of the present invention further comprises one or more skin care agents, soothing agents, deodorizing agent, probiotics, prebiotics, emollients, surfactants, thickening agents, sensates, polyols, humectants, polymers, silicones, enzymes, inorganic fillers, cationics, amphoterics, glycerides, esters, ethers, amino acids, polysaccharides, sugars, water or combinations thereof.

For instance, the cosmetic product of the present invention may comprise a skin care agent comprising a hydrophilic Phaeodactylum tricornutum extract. This cosmetic active, which has been described in WO 2020/178213 A1, has a soothing, comforting and inflammation-reducing effect on the skin and is therefore particularly advantageous for application to sensitive skin and sensitive skin areas. The disclosure of WO 2020/178213 A1 in this respect is herewith incorporated by reference.

The cosmetic product of the present invention may also comprise one or more prebiotics.

For instance, the cosmetic product of the present invention may comprise a prebiotic selected from the group consisting of fructans, galactans, pectin, beta-glucans, and combinations thereof. A number of prebiotics, which are able to balance the skin microbiome and are therefore advantageously included in the consumer product of the present invention, are described in WO 2020/052916 A1. The disclosure of WO 2020/052916 A1 and of the documents cited therein related to such prebiotics is herewith incorporated by reference.

The present invention also relates to a method of masking, reducing and/or preventing scalp malodour, said method comprising the step of applying the malodour counteracting fragrance composition or the consumer product of the present invention to the human scalp or hair.

In particular, the fragrance composition or consumer product may be applied to a location where body malodour is generally formed, for instance the underarm area, feet, scalp, crotch or groin.

The present invention is further illustrated by means of the following non-limiting examples:

### Example 1: Fragrance Compositions

A series of fragrance compositions were created, comprising Group A fragrance ingredients, Group B fragrance ingredients and other fragrance ingredients. These fragrance compositions are shown in Table 1 below. Fragrance compositions 1 through 10 are fragrance compositions according to the present invention. Fragrance compositions 11 to 13 are comparative examples.

### Example 2: Assessment of the Scalp Malodour Counteractancy

A 7×5 cm² piece of fabric consisting of 100% polyester (156 g/m²) was wetted by dipping it into cold water and pull it out one time in a jar containing clear water. The piece of fabric was handled with a clamp. 0.1 g of shampoo was applied on the wet fabric, which was folded in half and gently rubbed side against side for 30 seconds. The fabric was then rinsed by dipping it into cold water and pull it out five times, the excess water was removed by squeezing between two fingers and the fabric let dry on a grid for 120 minutes. 10 μL of malodour was deposed on a filter paper which was then put in a 125 ml brown jar. The piece of fabric was put on top of the filter paper imbibed with the malodour and the jar was sealed and let to equilibrate for 30 minutes. The jar was then opened 5 minutes prior to assessment by the panellist, in order to ventilate and dispel the headspace of the jar. The residual odour was then assessed directly in the jar.

Both malodour and fragrance intensity were assessed on a scale of 1-5 (1=barely noticeable, 2=weak, 3=medium, 4=strong and 5=very strong).

An overall scalp malodour counteraction (SMOC) score of 0 (poor) was given if the malodour intensity was higher than the fragrance intensity. An overall score of 1 (average) was given if the malodour intensity was similar to that of the fragrance intensity and a score of 2 (good) was given if the malodour intensity was lower than the fragrance intensity. The results are reported in Table 2:

TABLE 2
Scalp malodour counteraction performance of
the fragrance compositions of Example 1

| | Fragrance composition | | | | | | | | | | | | |
|---|---|---|---|---|---|---|---|---|---|---|---|---|---|
| | 1 | 2 | 3 | 4 | 5 | 6 | 7 | 8 | 9 | 10 | 11 | 12 | 13 |
| SMOC | 2 | 2 | 2 | 2 | 2 | 2 | 2 | 2 | 2 | 2 | 0 | 1 | 0 |
| score | | | | | | | | | | | | | |

TABLE 1

| | Fragrance Ingredients ↓ | | | | | | | | | | | | | |
|---|---|---|---|---|---|---|---|---|---|---|---|---|---|---|
| | Fragrance composition # | | | | | | | | | | | | | |
| | → | 1 | 2 | 3 | 4 | 5 | 6 | 7 | 8 | 9 | 10 | 11 | 12 | 13 |
| Group | AGRUMEX | 5.5 | 0 | 0 | 4.8 | 3.9 | 0 | 6.3 | 0 | 4.2 | 4.2 | 0 | 0 | 0.5 |
| A1 | AUBEPINE PARA | 0.1 | 0 | 0 | 0 | 0.4 | 0 | 1 | 0 | 0.4 | 0 | 0 | 0 | 0 |
| | CRESOL | | | | | | | | | | | | | |
| | BENZOIN | 0 | 0 | 0 | 0 | 0 | 0 | 0 | 2 | 0 | 0 | 0 | 0 | 0 |
| | CITRONELLOL | 0 | 1.5 | 5 | 1.1 | 0 | 0.1 | 2.5 | 0 | 0 | 0 | 0 | 0 | 0 |
| | DECALACTONE GAMMA | 3.5 | 0 | 3 | 1 | 0 | 0 | 3 | 0 | 0 | 0 | 0 | 4 | 0 |
| | FRUCTONE | 4.5 | 1 | 0 | 0 | 0 | 0 | 0 | 0 | 0 | 1 | 0 | 0 | 0 |
| | GALAXOLIDE | 0 | 0 | 0 | 0 | 6 | 4 | 3 | 0 | 0 | 10 | 0 | 0 | 0 |
| | HELIOTROPINE | 0 | 0 | 0 | 0 | 2.2 | 0 | 0.2 | 0 | 4 | 0 | 0 | 0 | 0 |
| | HYDROXYCITRONELLOL | 0 | 0 | 0 | 2.6 | 0 | 0 | 0 | 0 | 0 | 0 | 0 | 0 | 0 |
| | ISO E SUPER | 0 | 8 | 3 | 5 | 4 | 0 | 5 | 7 | 1 | 7 | 2 | 0 | 0.5 |
| | ISORALDEINE | 5.5 | 0 | 0 | 0 | 0 | 2.5 | 1 | 0 | 1 | 0 | 0 | 0 | 0 |
| | LILIAL | 7 | 0 | 0 | 0 | 0 | 0 | 0 | 0 | 0 | 0 | 0 | 0 | 2 |
| | MAYOL | 0 | 0 | 0 | 0 | 0 | 0.1 | 0 | 0 | 0 | 1.5 | 0 | 0 | 0 |
| | NOPYL ACETATE | 0 | 0 | 0 | 0 | 0 | 0 | 0 | 0 | 0.5 | 1.1 | 0 | 0 | 0 |
| | NYMPHEAL | 5 | 4.5 | 0 | 0 | 1.5 | 2 | 0 | 0 | 0.5 | 0 | 1.2 | 0 | 3 |
| | ROSYFOLIA | 0 | 0.4 | 0 | 0 | 0 | 0 | 0 | 0 | 0 | 0 | 0 | 3 | 0 |
| | TRIMOFIX O | 0 | 0 | 0 | 0.8 | 0 | 0 | 0 | 0 | 0 | 0 | 0 | 0 | 0 |
| Group | ACETOIN CRYSTALS | 0 | 0 | 0 | 0 | 0 | 0 | 0 | 0.6 | 0 | 0 | 0 | 0 | 0 |
| A2 | ALDEHYDE C 12 MNA | 0 | 0 | 0 | 0 | 0 | 0 | 0 | 1.2 | 0 | 0 | 0 | 1.5 | 0 |
| | AMBER CORE | 0 | 0 | 0.5 | 0 | 0 | 0 | 0 | 0 | 0 | 0.1 | 0 | 0 | 0 |
| | AMBROFIX | 0 | 1.5 | 0 | 1 | 0 | 0 | 0.2 | 0.3 | 0.2 | 0 | 0 | 0 | 0 |
| | ANISE STAR OIL | 0.2 | 0 | 0 | 0 | 0 | 0 | 0 | 0 | 0 | 0 | 0.2 | 0 | 0 |
| | BUTYL BUTYRO | 0 | 0 | 0 | 0 | 0 | 0.6 | 0 | 0 | 0 | 0 | 0 | 0 | 0 |
| | LACTATE | | | | | | | | | | | | | |
| | CALONE LIQUID | 0.1 | 0 | 0.3 | 0 | 0 | 0 | 0 | 0 | 0.05 | 0 | 0 | 0 | 0 |
| | CASSIS | 1 | 0 | 0 | 0 | 0 | 0 | 0 | 0 | 0 | 0 | 0 | 0 | 0 |
| | RECONSTITUTION | | | | | | | | | | | | | |
| | CEDARWOODOIL | 0.2 | 0 | 0 | 0 | 0 | 1.5 | 0 | 0 | 0 | 0 | 0 | 0 | 0 |
| | CEDRYL ACETATE | 0 | 0 | 7 | 0 | 0 | 0 | 0 | 1 | 0 | 0 | 0 | 0 | 0 |
| | DAMASCENONE GIV | 0 | 0 | 0 | 0 | 0 | 0 | 0 | 0 | 0 | 0.1 | 0 | 0 | 0 |
| Group | DAMASCONE ALPHA | 0.05 | 0.2 | 0 | 0 | 0 | 0 | 0 | 0 | 0 | 0 | 0.2 | 0 | 0 |
| A2 | DODECALACTONE | 0.6 | 0 | 0 | 0 | 0.4 | 0 | 0 | 0.3 | 0 | 0 | 0 | 0 | 0 |
| | DELTA | | | | | | | | | | | | | |
| | EBANOL | 0 | 0 | 0 | 0 | 0 | 0.9 | 0 | 0 | 0 | 0 | 0 | 0 | 0 |
| | FENNALDEHYDE | 0 | 0 | 0.2 | 0 | 0 | 0 | 0 | 0 | 0.2 | 0 | 0 | 0 | 0 |
| | FIXOLIDE | 0 | 0 | 0.8 | 0 | 0 | 0 | 0 | 0 | 0 | 0 | 0 | 0 | 0 |
| | ISOBUTAVAN | 0 | 0 | 0 | 0 | 0 | 0 | 0.1 | 0 | 0 | 0 | 0 | 0 | 0 |
| | MAHONIAL | 1 | 0 | 0 | 0 | 1 | 0 | 0 | 0 | 0 | 0 | 0 | 0 | 0 |
| | MALTOL | 0 | 0 | 0 | 0.5 | 0 | 0 | 0.1 | 0 | 0 | 0 | 0 | 0 | 0 |
| | MEFROSOL | 0 | 0 | 2 | 0 | 0 | 0 | 0 | 0 | 0 | 0 | 0 | 0 | 0 |
| | MENTHOL RACEMIC | 0 | 0 | 0 | 0 | 0 | 0 | 0 | 0 | 0 | 0 | 0.2 | 0 | 0 |
| | METHYL | 0 | 0 | 0 | 0 | 0 | 0 | 0.1 | 0 | 0.5 | 0 | 0 | 0 | 0.8 |
| | ANTHRANILATE | | | | | | | | | | | | | |
| | MUSCENONE | 0 | 0 | 0 | 0 | 0 | 0 | 0 | 0 | 0.1 | 0 | 0 | 0 | 0 |
| | NIRVANOLIDE | 0 | 0 | 0 | 0 | 0 | 0 | 0 | 0 | 0 | 0 | 0.2 | 0 | 0 |
| | PATCHOULI OIL | 0 | 0 | 0 | 0 | 0 | 0 | 0.7 | 0.2 | 0 | 0 | 0.4 | 0 | 0 |
| | RHUBOFIX | 0 | 0 | 0 | 1.1 | 0 | 0 | 0 | 0 | 0 | 0 | 0 | 0 | 0 |
| Group | ACETOPHENONE | 0.1 | 0 | 0 | 0 | 0 | 0 | 0 | 0 | 0 | 0 | 0 | 0 | 0 |
| A3 | AMBROCENIDE | 0.2 | 0 | 0 | 0 | 0 | 0 | 0 | 0.7 | 0.1 | 0 | 0 | 0 | 0.1 |
| | CAMPHOR | 0 | 0 | 0 | 0 | 0 | 0 | 0 | 0 | 0 | 0 | 0.1 | 0 | 0 |
| | CASSYRANE | 0 | 0.2 | 0 | 0 | 0.3 | 0 | 0 | 0 | 0 | 0 | 0 | 0 | 0 |
| | DIHYDRO EUGENOL | 0.3 | 0.1 | 0 | 0 | 0 | 0 | 0.1 | 0 | 0 | 0 | 0 | 0 | 0 |
| | ETHYL OENANTHATE | 0 | 0 | 0 | 0 | 0 | 0 | 0 | 0 | 0 | 0.15 | 0 | 0 | 0 |
| | JAVANOL | 0.05 | 0 | 0 | 0 | 0 | 0 | 0 | 0 | 0.15 | 0 | 0 | 0 | 0 |
| | METHYL LAITONE | 0 | 0 | 0 | 0 | 0.1 | 0 | 0 | 0.05 | 0 | 0 | 0 | 0 | 0 |
| | NEOFOLIONE | 0 | 0 | 0.1 | 0 | 0 | 0 | 0 | 0 | 0 | 0 | 0 | 0.3 | 0 |
| | NEROLEX | 0 | 0.1 | 0 | 0 | 0 | 1 | 0 | 0 | 0 | 0 | 0 | 0 | 0 |
| | NEROLIDOL | 0 | 0 | 0.1 | 0 | 0 | 0 | 0 | 0 | 0 | 0 | 0 | 0 | 0 |
| | VELOUTONE | 0 | 0 | 0.2 | 0 | 0 | 0 | 0 | 0 | 0 | 0 | 0 | 0 | 0 |
| Group | ACETAL CD | 0 | 0 | 0 | 0 | 0 | 0 | 0 | 0 | 0 | 0 | 0 | 0 | 1 |
| B | ALLYL CYCLOHEXYL | 0.3 | 0 | 0 | 0 | 0.6 | 0 | 0.5 | 0 | 0 | 0 | 0 | 1 | 0 |
| | PROPIONATE | | | | | | | | | | | | | |
| | AMYL CINNAMIC | 0 | 0 | 0 | 0 | 0 | 5 | 0 | 0 | 0 | 0 | 0 | 0 | 0 |
| | ALDEHYDE | | | | | | | | | | | | | |
| | BENZYL ACETATE | 0 | 0 | 0 | 2 | 1 | 0 | 1.3 | 0 | 1 | 3.5 | 0 | 6 | 0 |
| Group | CARVONE LAEVO | 0 | 0 | 0 | 0 | 0 | 0 | 0 | 0 | 0 | 0 | 0.6 | 0 | 0 |
| B | CETONE V | 0 | 0 | 0 | 0 | 0 | 0 | 0 | 0 | 0 | 0 | 0.4 | 0 | 0.4 |
| | CITRONELLYL ACETATE | 0 | 0 | 0 | 0 | 0 | 0 | 0 | 0 | 0 | 0.2 | 0 | 0 | 0.4 |
| | DIMETHYL BENZYL | 0 | 0 | 0 | 1.3 | 0 | 0 | 2 | 0 | 0.4 | 0 | 0 | 3 | 0 |
| | CARBINYL ACETATE | | | | | | | | | | | | | |
| | DIMETHYL BENZYL | 0 | 0 | 0 | 0 | 0 | 0 | 2 | 1 | 0 | 0 | 0 | 2.7 | 0 |
| | CARBINYL BUTYRATE | | | | | | | | | | | | | |
| | FLOROCYCLENE | 0 | 0 | 0 | 0 | 2 | 0 | 0 | 0 | 0 | 0 | 0 | 0 | 0 |
| | FLOROSA HC | 0.6 | 5 | 0 | 0 | 0 | 0 | 0 | 0 | 1.5 | 3 | 12.6 | 0 | 4 |
| | HEXYL CINNAMIC | 5 | 0 | 10 | 10 | 5.5 | 0 | 7 | 12.5 | 7.6 | 10 | 0 | 0 | 10 |
| | ALDEHYDE | | | | | | | | | | | | | |
| | IONONE BETA | 0 | 1.5 | 0 | 0 | 0 | 0 | 1.7 | 0 | 0 | 0 | 0 | 0 | 1.5 |
| | IRISONE ALPHA | 0 | 0 | 0 | 1 | 0 | 0 | 0 | 0 | 0 | 0 | 2 | 0 | 0 |
| | LEMONGRASS OIL | 0 | 0 | 0 | 0 | 0 | 0 | 0.1 | 0 | 0 | 0 | 0 | 0 | 0 |
| | METHYL ISOEUGENOL | 0 | 0 | 0 | 0 | 0 | 0 | 0 | 0 | 0 | 0 | 0.3 | 0 | 0 |
| | NERYL ACETATE | 0 | 0 | 0 | 0 | 0 | 0.5 | 0 | 0 | 0 | 0 | 0 | 0 | 0 |
| | OCTALACTONE GAMMA | 0 | 0 | 0 | 0 | 1 | 0 | 0 | 1 | 0 | 0 | 0 | 0 | 0 |
| | ORANGE OIL | 2.5 | 0 | 0 | 0 | 5 | 9.5 | 0 | 0 | 0.5 | 0.6 | 0 | 10 | 0 |
| | PHENOXY ETHYL | 0 | 0 | 0 | 0 | 0 | 0 | 2 | 0.5 | 0.4 | 0 | 0 | 0 | 1 |
| | ISOBUTYRATE | | | | | | | | | | | | | |
| | PHENYL ETHYL | 0 | 0 | 0 | 0 | 0 | 0 | 0 | 0 | 0 | 0.15 | 1.4 | 0 | 0 |
| | ACETATE | | | | | | | | | | | | | |
| | PHENYL ETHYL | 5.5 | 2.2 | 0.7 | 0 | 0 | 0 | 0.5 | 0 | 0.8 | 1 | 0 | 0 | 0 |
| | ALCOHOL | | | | | | | | | | | | | |
| | TRICYCLAL | 0 | 0 | 0 | 0 | 0 | 0 | 0 | 0 | 0 | 0 | 0 | 0.9 | 1 |
| Other | ALDEHYDE C 10 | 0.3 | 0 | 0 | 0 | 0 | 0 | 0 | 0 | 0 | 0 | 0.8 | 0 | 0 |
| | DECYLIC | | | | | | | | | | | | | |
| | ALLYL CAPROATE | 0 | 0 | 0 | 0 | 0 | 0 | 0.6 | 0 | 0 | 0 | 0 | 0 | 0 |
| | ALLYL OENANTHATE | 0 | 0 | 0.2 | 0 | 1.5 | 0 | 0.3 | 0 | 0 | 0 | 0 | 4 | 0 |
| | AMBRETTOLIDE | 0.6 | 0 | 0 | 0 | 0 | 0 | 0 | 0 | 0 | 0 | 0 | 0 | 0 |
| | AMYL SALICYLATE | 0 | 0 | 0 | 0 | 0 | 2 | 0 | 0 | 0.4 | 0 | 0 | 0 | 1.5 |
| | BENZYL SALICYLATE | 4 | 0 | 0 | 4 | 0 | 0 | 6 | 0 | 0 | 5 | 0 | 0 | 11 |
| | BERGAMOT OIL | 0 | 6 | 0 | 0 | 0 | 0 | 0 | 0 | 0 | 0 | 0 | 0 | 0 |
| | BICYCLO | 0 | 0 | 0 | 0 | 0 | 0 | 0 | 0 | 0.2 | 0 | 0 | 0 | 0 |
| | NONALACTONE | | | | | | | | | | | | | |
| | BUTYL CYCLOHEXYL | 0 | 0 | 0 | 0 | 11 | 0 | 0 | 0 | 0 | 0 | 0 | 0 | 0 |
| | ACETATE PARA | | | | | | | | | | | | | |
| | CASHMERAN | 0 | 0 | 0 | 0 | 6 | 0 | 0 | 5 | 0.6 | 2 | 2 | 0 | 0 |
| | CEDRYL METHYL | 0 | 0 | 0 | 5 | 0 | 0 | 0 | 5 | 0.5 | 0 | 0 | 0 | 0 |
| | ETHER | | | | | | | | | | | | | |
| | CINNAMIC ALCOHOL | 0 | 0 | 0 | 2 | 0 | 0 | 0 | 0 | 0 | 0 | 0 | 0 | 0 |
| Other | CORIANDER SEED OIL | 0 | 2 | 0 | 0 | 0 | 0 | 0 | 0 | 0 | 0 | 0 | 0 | 0 |
| | COUMARIN PURE | 1.5 | 0 | 0 | 0 | 10 | 0 | 4 | 3.3 | 0.5 | 0 | 0 | 0.3 | 0 |
| | CYCLAMEN ALDEHYDE | 0.2 | 0 | 2 | 1 | 0 | 0 | 0.5 | 0 | 0 | 0 | 0 | 0 | 0 |
| | DIETHYL MALONATE | 0 | 0 | 0 | 0 | 0 | 0 | 0 | 0 | 0 | 0 | 0 | 5 | 0 |
| | DIHYDRO MYRCENOL | 0 | 0 | 0 | 0 | 0 | 0 | 1.6 | 0 | 0 | 3.5 | 4 | 0 | 0 |
| | DIPROPYLENE GLYCOL | 8 | 1.1 | 0 | 6.6 | 3.1 | 3.9 | 0 | 31.4 | 26.3 | 5 | 26.3 | 3.5 | 15 |
| | ETHYL ACETATE | 1 | 0.6 | 0 | 0 | 0 | 0 | 0 | 0 | 0 | 0 | 0 | 0 | 0 |
| | ETHYL LINALOOL | 2.3 | 0 | 0 | 1.5 | 0 | 0 | 0 | 0 | 3 | 0 | 0 | 0 | 0 |
| | ETHYL METHYL-2- | 2.2 | 0 | 0 | 0 | 0 | 0 | 1 | 0 | 0 | 0 | 0 | 1 | 1 |
| | BUTYRATE | | | | | | | | | | | | | |
| | ETHYL VANILLIN | 1.5 | 0 | 0 | 0 | 0 | 0 | 4 | 3 | 4.5 | 0 | 2.1 | 0 | 0 |
| | ETHYLENE | 3.4 | 8 | 0 | 12 | 0 | 2 | 4 | 0 | 4.8 | 0 | 0 | 0 | 0 |
| | BRASSYLATE | | | | | | | | | | | | | |
| | FLORHYDRAL | 0 | 0 | 0 | 0 | 0 | 0 | 0 | 0 | 2 | 1 | 2 | 0 | 1 |
| | GERANIOL | 0 | 3 | 0 | 1 | 3 | 3 | 0 | 0 | 0 | 0 | 3 | 10 | 0.2 |
| | HABANOLIDE | 0.8 | 4.1 | 8 | 0 | 0 | 2 | 0 | 0 | 0 | 0 | 0 | 4 | 10 |
| | HEDIONE | 7 | 13 | 20 | 10 | 5 | 7 | 9 | 0 | 8 | 10 | 8 | 10 | 8 |
| | HELVETOLIDE | 0 | 0 | 0 | 2 | 6 | 0 | 0 | 0 | 0 | 10 | 0 | 0 | 0 |
| | HEXYL ACETATE | 1 | 0 | 0 | 1 | 2.8 | 0 | 1 | 0 | 1 | 0 | 0 | 2 | 1.8 |
| | HEXYL SALICYLATE | 0 | 0 | 0 | 2 | 2 | 0 | 0 | 0 | 1 | 0 | 1 | 0 | 0 |
| | LEMON OIL | 1.3 | 5 | 0 | 0 | 0 | 0 | 0 | 0 | 0 | 3 | 0 | 0 | 0 |
| | LIFFAROME | 0 | 0 | 0 | 0 | 0 | 0 | 0.2 | 0 | 0 | 0 | 0 | 0 | 0 |
| | LINALOOL | 0 | 0 | 8 | 0 | 2 | 10 | 2.8 | 0 | 4 | 6 | 10 | 8 | 10 |
| | LINALYL ACETATE | 0 | 4 | 2 | 2.2 | 0 | 10 | 0 | 0 | 4.1 | 3.2 | 3.8 | 0 | 0 |
| | MANZANATE | 0 | 0 | 0 | 0 | 0.7 | 0 | 0.3 | 0 | 0 | 0 | 0 | 1.5 | 0.4 |
| | MUSK C14 | 3.5 | 2 | 0 | 0 | 0 | 0 | 0 | 0 | 0 | 0 | 1.8 | 0 | 0 |
| | NOOTKATONE | 0 | 0 | 0 | 0.5 | 0 | 0 | 0 | 0 | 0 | 0 | 0 | 0 | 0 |
| | NUTMEG OIL | 0 | 1 | 0 | 0 | 0 | 0 | 0 | 0 | 0 | 0 | 0 | 0 | 0 |
| | LIMONENE | 0.4 | 7 | 8 | 0 | 0 | 20.9 | 2 | 0 | 2 | 7 | 3 | 0 | 0 |
| | PEACH PURE | 4 | 0 | 0 | 1 | 2 | 0 | 4.5 | 0 | 0 | 0 | 0 | 0 | 0 |
| | PINE OIL | 1.1 | 5 | 0 | 2.3 | 0 | 0 | 0 | 0 | 0 | 0 | 0 | 0 | 0 |
| | PRUNOLIDE | 0 | 0 | 0 | 0 | 5 | 0 | 3 | 8 | 0 | 0 | 0 | 0 | 0 |
| | RADJANOL | 0 | 0 | 0 | 0 | 0 | 0 | 4 | 2 | 1.5 | 0 | 0 | 0 | 0 |
| Other | RASPBERRY KETONE | 4 | 0 | 0 | 3.7 | 1 | 0 | 2 | 5 | 0.8 | 0 | 2 | 0 | 0 |
| | ROSE OXIDE | 0 | 0 | 0 | 0 | 0 | 0 | 0.1 | 0 | 0 | 0 | 0 | 0 | 0 |
| | SANDALORE | 0 | 0 | 0 | 1.5 | 0 | 0 | 0 | 6.5 | 3 | 0.5 | 0 | 0 | 0 |
| | SERENOLIDE | 0 | 0 | 0 | 2 | 0 | 0 | 0 | 0 | 0 | 0 | 0 | 0 | 0 |
| | STYRALLYL ACETATE | 1.3 | 12 | 0 | 0 | 0 | 0 | 2 | 0 | 0.2 | 0 | 0 | 2 | 2 |
| | TERPINEOL | 0 | 0 | 0.4 | 0 | 0 | 1.5 | 0.8 | 0 | 0 | 0 | 0 | 0.7 | 2 |
| | TETRAHYDRO- | 1 | 0 | 12 | 2.5 | 2 | 10 | 5.5 | 2.5 | 4.5 | 0 | 5.4 | 10 | 2 |
| | LINALOOL | | | | | | | | | | | | | |
| | TRIETHYL CITRATE | 0 | 0 | 4.5 | 1 | 0 | 0 | 0 | 0 | 0 | 0 | 1 | 4.5 | 6 |
| | TROPIONAL | 0.2 | 0 | 2 | 0 | 0 | 0 | 0 | 0 | 2 | 0.2 | 2 | 1.1 | 1.2 |
| | UNDECAVERTOL | 0.6 | 0 | 0 | 0 | 0 | 0 | 0.4 | 0 | 0 | 0 | 0 | 0 | 0.7 |
| | VANILLIN | 0 | 0 | 0 | 3 | 2 | 0 | 0 | 0 | 0 | 0 | 0 | 0 | 0 |
| | Sum Group A1 (W_{A1}) | 31.1 | 15.4 | 11 | 15.3 | 18 | 8.7 | 22 | 9 | 11.6 | 24.8 | 3.2 | 7 | 6 |
| | Sum Group A2 (W_{A2}) | 3.15 | 1.7 | 10.8 | 15.3 | 1.4 | 3 | 1.2 | 3.6 | 1.05 | 0.2 | 1.2 | 1.5 | 0.8 |
| | Sum Group A3 (W_{A3}) | 0.65 | 0.4 | 0.4 | 2.6 | 0.4 | 1 | 0.1 | 0.75 | 0.25 | 0.15 | 0.1 | 0.3 | 0.1 |
| | Sum Group B (W_{B}) | 13.9 | 8.7 | 10.7 | 14.3 | 15.1 | 15.0 | 17.1 | 15.0 | 12.2 | 18.5 | 17.3 | 23.6 | 19.3 |
| | RATIO A/B | 2.5 | 2.0 | 2.1 | 2.3 | 1.3 | 0.8 | 1.4 | 0.9 | 1.1 | 1.4 | 0.3 | 0.4 | 0.4 |
| | $\frac{{W}_{A 1} + 10 {W}_{A 2} + 100 {W}_{A 3}}{{W}_{B}}$ | 9.2 | 8.3 | 14.9 | 30.0 | 4.8 | 9.2 | 2.6 | 8.0 | 3.9 | 2.9 | 1.5 | 2.2 | 1.2 |

The results of Table 2 demonstrate the superiority of the fragrance compositions according to the present invention in counteracting scalp malodour.

### Example 3: Assessment of the Scalp Malodour Counteractancy of Fragrance Ingredients

In a first set of experiments, the scalp malodour counteractancy of 173 fragrance ingredients was evaluated by panel of 5 experts.

The evaluation was performed in the gas phase, using a MiniVAS™ device (ex Givaudan SA). This device is a regulating means that comprises a plurality of channels adapted to convey a gas (more particularly air) through cartridges, each cartridge containing a fragrance ingredient adsorbed onto an adsorbent. The air flows coming from the different cartridges are admixed and conveyed to the user's nose through a tubing fitted with an olfactometer funnel. Thus, by opening and closing channels and maintaining these opened and closed, respectively, for predetermined times, the regulating means can determine which fragrance ingredients and how much thereof are conveyed and when, and thus alter the nature of the fragrance perceived by the user. A description of the device can be found in WO2014154909A1, page 2, line 11 to page 4, line 7, and in H. R. Gygax et al. “Evolution of Real Time Aroma Creation at Givaudan”, poster presented at the 12th Weurman Flavour Research Symposium. July 2008. Interlaken, Switzerland; DOI: 10.13140/2.1.1924.0641.

For the sake of the present evaluation, one cartridge contained the scalp malodour (in the form of a model malodour) and each fragrance ingredient was assigned to another cartridge. The malodour counteractancy of each fragrance ingredient was evaluated separately against the scalp malodour. A constant air flow rate of 50 ml/min was used for all measurements. The perceived malodour intensity, as well as the fragrance intensity, were ranked on a scale of 1-5 (1=barely noticeable, 2=weak, 3=medium, 4=strong and 5=very strong).

In a second set of experiments, the impact of these fragrance ingredients on the scalp malodour counteractancy of a fragrance composition was assessed. The scalp malodour counteractancy of a series of 80 fragrance compositions was assessed by performing the evaluation protocol described in Example 2. As in Example 2, fragrance compositions having a high malodour counteractancy were those compositions for which the fragrance intensity was higher than the malodour intensity, fragrance compositions having a low malodour counteractancy were those compositions for which the fragrance intensity was lower than the malodour intensity. Ingredients that were statistically more present in fragrances having a high malodour counteractancy were assigned a positive status, whereas ingredients that were statistically more present in compositions having a low malodour counteractancy were assigned a negative status.

Finally, fragrance ingredients having either malodour counteractancy properties when used alone or a positive impact on the malodour counteractancy of full fragrance compositions were grouped according to their odour intensity.

The odour intensity of a fragrance ingredient may be measured by olfactometry. Typically, the intensity is assessed by a panel of people who score the olfactive impact of an ingredient on a scale as a function of the concentration of this ingredient in the air. A concentration-intensity curve is obtained which may typically have a sigmoid shape, with a low intensity region and a high intensity plateau region and an intermediate region between these two regions where the intensity increases steeply (threshold). The odour intensity of a fragrance ingredient is related to the position and the steepness of the sigmoid, as well as to the height of the high intensity region. The lower the concentration of the sigmoid threshold, the steeper the sigmoid and the higher the high intensity plateau, the more intense is an ingredient. On the other hand, the dosage of a fragrance ingredient in a perfume depends on the odour intensity of this ingredient: high intensity ingredients are typically used at a low level in a perfume. Thus, the dosage at which an ingredient is typically used in perfumery may also be considered as a suitable measurement of the intensity of this ingredient.

Hence, the fragrance ingredients having a positive impact on scalp malodour counteractancy were grouped into Groups A1, A2 and A3, depending on their intensity and, therefore, on their average dosage in fragrance compositions.

The results of the above assessments are reported in Table 3 and Table 4 below.

As apparent from Table 3, some fragrance ingredients show a positive impact on the malodour counteractancy of full fragrance composition, although they are inactive when used alone against the scalp malodour. This result was not anticipated from the state of the art and hints at synergies between certain fragrance ingredients in a fragrance composition.

Fragrance ingredients being neutral with respect to malodour counteractancy are not listed here.

TABLE 3
Fragrance ingredients having positive scalp malodour counteractancy (MOC).

| | | | Impact on | | |
|---|---|---|---|---|---|
| | | Scalp MOC of | scalp MOC | | |
| | MO | ingredient | of fragrance | Ingredient | |
| Fragrance ingredient | intensity | alone | composition | intensity | Group |
| 3-hydroxybutan-2-one | 4 | Depresses | Positive | High | A3 |
| | | MO's fatty | | | |
| | | smell | | | |
| 1-phenylethanone | 5 | Highly | Positive | High | A3 |
| | | perfoming | | | |
| 2-(tert-butyl)cyclohexyl acetate | 2 | Combines well | Positive | Low | A1 |
| | | with MO and | | | |
| | | turns it into | | | |
| | | apple note | | | |
| 2-methylundecanal | 3.5 | Good | Positive | Medium | A2 |
| | | coverage | | | |
| 1-((2-(tert- | 2.3 | Coverage | Positive | Medium | A2 |
| butyl)cyclohexyl)oxy)butan-2-ol | | | | | |
| (4aR,5R,7aS,9R)-Octahydro- | 4 | Depresses | Positive | High | A3 |
| 2,2,5,8,8,9a-hexamethyl-4H-4a,9- | | fatty smell of | | | |
| methanoazuleno[5,6-d]-1,3-dioxole | | MO | | | |
| (3aR,5aS,9aS,9bR)-3a,6,6,9a- | 1 | No effect | Positive | Medium | A2 |
| tetramethyl-2,4,5,5a,7,8,9,9b- | | | | | |
| octahydro-1H-benzo[e][1]benzofuran | | | | | |
| (E)-1-methoxy-4-(prop-1-en-1- | 4 | Combines well | Positive | Medium | A2 |
| yl)benzene | | with MO, turns | | | |
| | | it into jasmine- | | | |
| | | anise note | | | |
| Anise star oil | 3 | Combines well | Positive | High | A3 |
| | | with MO, turns | | | |
| | | it into anise | | | |
| | | note | | | |
| 4-methoxybenzaldehyde | 3 | Combines well | Positive | Low | A1 |
| | | with MO, turns | | | |
| | | it into floral | | | |
| | | note | | | |
| 7-isopentyl-2H- | 4.5 | Combines well | Positive | High | A3 |
| benzo[b][1,4]dioxepin-3(4H)-one | | with MO, turns | | | |
| | | it into floral | | | |
| | | note | | | |
| Benzoin resinoid | 4 | Highly | Positive | Low | A1 |
| | | perfoming | | | |
| 1-butoxy-1-oxopropan-2-yl | 1 | No effect | Positive | Medium | A2 |
| butanoate | | | | | |
| 8-methyl-1,5-benzodioxepin-3-one | 3.5 | Good | Positive | Medium | A2 |
| | | coverage | | | |
| 6-methoxy-2,6-dimethyloctanal | 3 | Good | Positive | High | A3 |
| | | coverage | | | |
| (1S,4S)-1,7,7- | 4.5 | Highly | Positive | High | A3 |
| trimethylbicyclo[2.2.1]heptan-2-one | | perfoming | | | |
| Blackcurrent reconstitution | 2 | Combines well | Positive | High | A3 |
| | | with MO and | | | |
| | | turns it into | | | |
| | | cassis-green | | | |
| | | note | | | |
| 5-tert-butyl-2-methyl-5-propyl-2H- | 4 | Combines well | Positive | High | A3 |
| furan | | with MO and | | | |
| | | turns it into | | | |
| | | cassis-green | | | |
| | | note | | | |
| Cedar wood oil | 1 | No effect | Positive | Low | A1 |
| (1S,6R,8aR)-1,4,4,6- | 1 | No effect | Positive | Low | A1 |
| tetramethyloctahydro-1H-5,8a- | | | | | |
| methanoazulen-6-yl acetate | | | | | |
| (1S,6R,8aR)-1,4,4,6- | 1 | No effect | Positive | Low | A1 |
| tetramethyloctahydro-1H-5,8a- | | | | | |
| methanoazulen-6-yl acetate | | | | | |
| 3,7-dimethyloct-6-enal | 3 | Good | Positive | High | A3 |
| | | coverage | | | |
| 3,7-dimethyloct-6-en-1-ol | 2.5 | Combines well | Positive | Low | A1 |
| | | with MO and | | | |
| | | turns it into | | | |
| | | floral-animalic | | | |
| | | note | | | |
| (4-isopropylphenyl)methanol | 2 | Combines well | Positive | High | A3 |
| | | with MO and | | | |
| | | turns it into | | | |
| | | spicy note | | | |
| cyclohexyl 2-hydroxybenzoate | 1 | No effect | Positive | Low | A1 |
| (E)-1-(2,6,6-trimethylcyclohexa-1,3- | 2.8 | Good | Positive | High | A3 |
| dien-1-yl)but-2-en-1-one | | coverage, but | | | |
| | | increased | | | |
| | | fattiness | | | |
| (E)-1-(2,6,6-trimethylcyclohex-2-en- | 3.7 | Combines well | Positive | Medium | A2 |
| 1-yl)but-2-en-1-one | | with MO and | | | |
| | | turns it into | | | |
| | | fruity note | | | |
| (E)-1-(2,6,6-trimethyl-1- | 2.8 | Coverage | Positive | High | A3 |
| cyclohexenyl)but-2-en-1-one | | | | | |
| 5-hexyloxolan-2-one | 3 | Combines well | Positive | Low | A1 |
| | | with MO and | | | |
| | | turns it into | | | |
| | | fruity note | | | |
| 2-methoxy-4-propylphenol | 2 | Combines well | Positive | High | A3 |
| | | with MO and | | | |
| | | turns it into | | | |
| | | spicy note | | | |
| 6-heptyltetrahydro-2H-pyran-2-one | 3 | Combines well | Positive | Low | A1 |
| | | with MO and | | | |
| | | turns it into | | | |
| | | fruity note | | | |
| (E)-4-((3aS,7aS)-hexahydro-1H-4,7- | 2.5 | Combines well | Positive | High | A3 |
| methanoinden-5(6H)-ylidene)butanal | | with MO and | | | |
| | | turns it into | | | |
| | | clean note | | | |
| (E)-3-methyl-5-(2,2,3- | 4.3 | Very good | Positive | Medium | A2 |
| trimethylcyclopent-3-en-1-yl)pent-4- | | coverage | | | |
| en-2-ol | | | | | |
| ethyl 2-methylpropanoate | 1 | No effect | Positive | Medium | A2 |
| ethyl octanoate | 1 | No effect | Positive | High | A3 |
| ethyl heptanoate | 1 | No effect | Positive | High | A3 |
| 4-allyl-2-methoxyphenol | 4 | Combines well | Positive | High | A3 |
| | | with MO and | | | |
| | | turns it into | | | |
| | | resinous-spicy | | | |
| | | note | | | |
| 3-(4-methoxyphenyl)-2- | 1 | No effect | Positive | Medium | A2 |
| methylpropanal | | | | | |
| 1-(3,5,5,6,8,8-hexamethyl-5,6,7,8- | 3 | Good | Positive | Medium | A2 |
| tetrahydronaphthalen-2-yl)ethanone | | coverage | | | |
| ethyl 2-(2-methyl-1,3-dioxolan-2- | 2.3 | Combines well | Positive | Low | A1 |
| yl)acetate | | with MO and | | | |
| | | turns it into | | | |
| | | green anise | | | |
| | | note | | | |
| 4,6,6,7,8,8-hexamethyl-1,3,4,6,7,8- | 3 | Good | Positive | Medium | A2 |
| hexahydrocyclopenta[g]isochromene | | coverage | | | |
| Geranium oil | 4 | Very good | Positive | Medium | A2 |
| | | coverage | | | |
| benzo[d][1,3]dioxole-5-carbaldehyde | 3 | Combines well | Positive | Low | A1 |
| | | with MO and | | | |
| | | turns it into a | | | |
| | | floral note | | | |
| 7-hydroxy-3,7-dimethyloctanal | 2 | Medium | Positive | Medium | A2 |
| | | coverage | | | |
| 3,7-dimethyloctane-1,7-diol | 1 | No effect | Positive | Medium | A2 |
| 1H-indole | 4 | Good | Positive | High | A3 |
| | | coverage and | | | |
| | | increase the | | | |
| | | acceptability of | | | |
| | | MO | | | |
| 1-(2,3,8,8-tetramethyl- | 1 | No effect | Positive | Low | A1 |
| 1,2,3,4,5,6,7,8- | | | | | |
| octahydronaphthalen-2-yl)ethanone | | | | | |
| 4-formyl-2-methoxyphenyl 2- | 1.5 | Combines well | Positive | High | A3 |
| methylpropanoate | | with MO to | | | |
| | | make it | | | |
| | | sweeter | | | |
| (E)-2-methoxy-4-(prop-1-en-1- | 4.5 | Very | Positive | High | A3 |
| yl)phenol | | performing, | | | |
| | | strong clove | | | |
| | | note | | | |
| (E)-1-(2,6,6-trimethylcyclohex-2-en- | 3 | Combines well | Positive | Low | A1 |
| 1-yl)pent-1-en-3-one | | with MO and | | | |
| | | turns it into | | | |
| | | floral-orris note | | | |
| (E)-3-methyl-4-(2,6,6- | 3 | Combines well | Positive | Medium | A2 |
| trimethylcyclohex-2-en-1-yl)but-3- | | with MO and | | | |
| en-2-one | | turns it into | | | |
| | | floral-orris note | | | |
| 1-methyl-2-((1,2,2- | 3.3 | Good | Positive | High | A3 |
| trimethylbicyclo[3.1.0]hexan-3- | | coverage | | | |
| yl)methyl)cyclopropyl)methanol | | | | | |
| 3-(4-(tert-butyl)phenyl)-2- | 2.8 | Combines well | Positive | Low | A1 |
| methylpropanal | | with MO and | | | |
| | | turns it into | | | |
| | | marine note | | | |
| (4E)-9-hydroxy-5,9-dimethyl-4- | 1 | No effect | Positive | Medium | A2 |
| decenal | | | | | |
| 3-hydroxy-2-methyl-4H-pyran-4-one | 4 | Very good | Positive | Medium | A2 |
| | | coverage | | | |
| (4-isopropylcyclohexyl)methanol | 3 | Combines well | Positive | Medium | A2 |
| | | with MO and | | | |
| | | turns it into | | | |
| | | floral-marine | | | |
| | | note | | | |
| 3-methyl-5-phenylpentan-1-ol | 2 | Low coverage | Positive | Medium | A2 |
| | | when used | | | |
| | | alone | | | |
| 2-isopropyl-5-methylcyclohexanol | 1.2 | Almost no | Positive | Medium | A2 |
| | | effect when | | | |
| | | used alone | | | |
| mercapto-para-menthan-3-one | 1 | No effect | Positive | High | A3 |
| methyl 2-aminobenzoate | 4.3 | Very good | Positive | Medium | A2 |
| | | coverage | | | |
| 8-methyl-1-oxaspiro[4.5]decan-2- | 2.7 | Combines well | Positive | High | A3 |
| one | | with MO and | | | |
| | | turns it into | | | |
| | | sweet-cooked | | | |
| | | note | | | |
| methyl non-2-ynoate | 1.8 | Poor, | Positive | High | A3 |
| | | mushroom | | | |
| | | note | | | |
| (E)-3-methyl-4-(2,6,6- | 3 | Good | Positive | Medium | A2 |
| trimethylcyclohex-1-en-1-yl)but-3- | | coverage | | | |
| en-2-one | | | | | |
| (Z)-3-methylcyclopentadec-5-enone | 2.1 | Combines well | Positive | Medium | A2 |
| | | with MO and | | | |
| | | turns it into dry | | | |
| | | note | | | |
| (E)-methyl non-2-enoate | 1 | No effect | Positive | High | A3 |
| (2Z)-3,7-dimethylocta-2,6-dien-1-ol | 1 | No effect | Positive | High | A3 |
| (E)-3,7,11-trimethyldodeca-1,6,10- | 1 | No effect | Positive | High | A3 |
| trien-3-ol | | | | | |
| (E)-13-methyloxacyclopentadec-10- | 2 | Low coverage | Positive | Medium | A2 |
| en-2-one | | when used | | | |
| | | alone | | | |
| 2-(6,6-dimethylbicyclo[3.1.1]hept-2- | 2 | Low coverage | Positive | Low | A1 |
| en-2-yl)ethyl acetate | | when used | | | |
| | | alone | | | |
| 3-(4-(2-methylpropyl)-2- | 2.8 | Combines well | Positive | Low | A1 |
| methylphenyl)propanal | | with MO and | | | |
| | | turns it into | | | |
| | | marine-lily of | | | |
| | | the valley note | | | |
| Patchouli oil | 4.3 | Very good | Positive | Low | A1 |
| | | coverage but | | | |
| | | foul note when | | | |
| | | used alone | | | |
| 3,7-dimethyloctan-1-ol | 4.3 | Combines well | Positive | High | A3 |
| | | with MO and | | | |
| | | turns it into | | | |
| | | floral-lily of the | | | |
| | | valley note | | | |
| (2E,5E)-5,6,7-trimethylocta-2,5-dien- | 1.5 | Almost no | Positive | High | A3 |
| 4-one | | effect when | | | |
| | | used alone | | | |
| (2R,8aS)-3′,6-dimethyl- | 1 | No effect | Positive | Medium | A2 |
| 3,4,4a,5,8,8a-hexahydro-1H- | | | | | |
| spiro[1,4-methanonaphthalene-2,2′- | | | | | |
| oxirane] | | | | | |
| 1-methyl-2-(5-methylhex-4-en-2- | 2.7 | Combines well | Positive | Low | A1 |
| yl)cyclopropylmethanol | | with MO and | | | |
| | | turns it into | | | |
| | | floral-rosy note | | | |
| 2-(4-Methyl-1,3-thiazol-5-yl)ethanoL | 1 | No effect | Positive | High | A3 |
| (E)-6-ethyl-3-methyloct-6-en-1-ol | 3 | Combines well | Positive | High | A3 |
| | | with MO and | | | |
| | | turns it into | | | |
| | | floral-lily of the | | | |
| | | valley note | | | |
| 1-((2E,5Z,9Z)-2,7,8- | 1 | No effect | Positive | High | A3 |
| trimethylcyclododeca-2,5,9-trien-1- | | | | | |
| yl)ethanone | | | | | |
| 2-ethoxy-4-methylphenol | 2.8 | Medium | Positive | High | A3 |
| | | coverage | | | |
| 2,2,5-trimethyl-5- | 3.5 | Good | Positive | High | A3 |
| pentylcyclopentanone | | coverage but | | | |
| | | fatty note | | | |
| | | when used | | | |
| | | alone | | | |

TABLE 4
Fragrance ingredients having negative impact on scalp malodour counteractancy (MOC).

| | | | Impact on scalp | |
|---|---|---|---|---|
| | MO | Scalp MOC of | MOC of fragrance | |
| Fragrance ingredient | intensity | ingredient alone | composition | Group |
| (2-benzyl-1,3-dioxolan-4-yl)methanol | 1 | No effect when | Negative | B |
| | | used alone | | |
| undecanal | 1 | Enhances MO | Negative | B |
| prop-2-enyl 3-cyclohexylpropanoate | 1.5 | Almost no effect | Negative | B |
| | | when used alone | | |
| Pineapple reconstitution | 1 | No effect when | Negative | B |
| | | used alone | | |
| benzyl acetate | 0 | Enhances MO | Negative | B |
| benzyl propanoate | 1 | Almost no effect | Negative | B |
| | | when used alone | | |
| (5R)-2-methyl-5-prop-1-en-2- | 0 | Enhances MO | Negative | B |
| ylcyclohex-2-en-1-one | | | | |
| (E)-1-(2,6,6-trimethylcyclohex-2-en-1- | 3 | Good coverage | Negative | B |
| yl)hepta-1,6-dien-3-one | | | | |
| 3,7-dimethyloct-6-en-1-yl acetate | 2 | Low coverage | Negative | B |
| 2,4-dimethylcyclohex-3-ene-1- | 1 | Enhances oily | Negative | B |
| carbaldehyde | | rancid note of MO | | |
| allyl 2-(cyclohexyloxy)acetate | 4.3 | Very high | Negative | B |
| | | coverage, but | | |
| | | pungent smell | | |
| 4-(2,6,6-trimethylcyclohex-1-en-1- | 1 | No effect when | Negative | B |
| yl)butan-2-one | | used alone | | |
| 2-methyl-1-phenylpropan-2-ol | 1 | No effect when | Negative | B |
| | | used alone | | |
| 2-methyl-1-phenylpropan-2-yl acetate | 3 | Partial | Negative | B |
| | | neutralization of | | |
| | | MO when used | | |
| | | alone | | |
| 2-methyl-1-phenylpropan-2-yl | 3 | Partial | Negative | B |
| butanoate | | neutralization of | | |
| | | MO when used | | |
| | | alone | | |
| 4,7-dimethyloct-6-en-3-one | 4 | High coverage | Negative | B |
| | | when used alone | | |
| (2E,4Z)-ethyl deca-2,4-dienoate | 1 | No effect when | Negative | B |
| | | used alone | | |
| ethyl 2,6,6-trimethylcyclohexa-1,3- | 1 | No effect when | Negative | B |
| diene-1-carboxylate | | used alone | | |
| 4-allyl-2-methoxyphenyl acetate | 1 | No effect when | Negative | B |
| | | used alone | | |
| (3aR,6S,7aS)-3a,4,5,6,7,7a- | 1.3 | Almost no effect | Negative | B |
| hexahydro-1H-4,7-methanoinden-6-yl | | when used alone | | |
| propanoate | | | | |
| 2,4,6-trimethyl-4-phenyl-1,3-dioxane | 1 | No effect when | Negative | B |
| | | used alone | | |
| tetrahydro-4-methyl-2-(2- | 2.2 | Low coverage | Negative | B |
| methylpropyl)-2H-pyran-4-ol | | | | |
| 2-ethyl-4-hydroxy-5-methylfuran- | 4.5 | Turns MO into | Negative | B |
| 3(2H)-one | | burnt caramel | | |
| (Z)-hex-3-en-1-yl benzoate | 2 | Low coverage | Negative | B |
| (E)-2-benzylideneoctanal | 1.2 | Enhances MO fatty | Negative | B |
| | | note | | |
| (E)-4-(2,6,6-trimethylcyclohex-1-en-1- | 1 | No effect when | Negative | B |
| yl)but-3-en-2-one | | used alone | | |
| (E)-4-(2,6,6-trimethyl-1-cyclohex-2- | 4.5 | Very high | Negative | B |
| enyl)but-3-en-2-one | | coverage, but | | |
| | | pungent smell | | |
| (3aR,6S,7aS)-3a,4,5,6,7,7a- | 3.8 | High coverage, but | Negative | B |
| hexahydro-1H-4,7-methanoinden-6-yl | | unpleasant off-note | | |
| acetate | | | | |
| (Z)-3-methyl-2-(pent-2-en-1- | 1.8 | Low coverage | Negative | B |
| yl)cyclopent-2-enone | | | | |
| 3-butyl-5-methyltetrahydro-2H-pyran- | 1 | No effect when | Negative | B |
| 4-yl acetate | | used alone | | |
| Lemongrass oil | 1 | No effect when | Negative | B |
| | | used alone | | |
| Lime oil | 1 | No effect when | Negative | B |
| | | used alone | | |
| 1-((1S,8aS)-1,4,4,6-tetramethyl- | 1 | No effect when | Negative | B |
| 2,3,3a,4,5,8-hexahydro-1H-5,8a- | | used alone | | |
| methanoazulen-7-yl)ethanone | | | | |
| methyl 2-hexyl-3-oxocyclopentane-1- | 1 | No effect when | Negative | B |
| carboxylate | | used alone | | |
| (E)-1,2-dimethoxy-4-(prop-1-en-1- | 3.3 | Partial | Negative | B |
| yl)benzene | | neutralization of | | |
| | | MO when used | | |
| | | alone | | |
| 4-(4-methylpent-3-en-1-yl)cyclohex-3- | 1 | No effect when | Negative | B |
| enecarbaldehyde | | used alone | | |
| (Z)-3,7-dimethylocta-2,6-dien-1-yl | 1 | No effect when | Negative | B |
| acetate | | used alone | | |
| 6-propyltetrahydro-2H-pyran-2-one | 3.8 | High coverage of | Negative | B |
| | | MO, but buttery off- | | |
| | | note | | |
| 5-butyloxolan-2-one | 3.8 | Combines with MO | Negative | B |
| | | but butyric off-note | | |
| Orange oils | 1 | No effect when | Negative | B |
| | | used alone | | |
| 1-(2-naphtalenyl)-ethanone | 1 | No effect when | Negative | B |
| | | used alone | | |
| 2-methyl-4-propyl-1,3-oxathiane | 1 | No effect when | Negative | B |
| | | used alone | | |
| Passion fruit reconstitution | 1 | No effect when | Negative | B |
| | | used alone | | |
| 2-cyclohexylidene-2- | 1 | No effect when | Negative | B |
| phenylacetonitrile | | used alone | | |
| 2-(phenoxy)ethyl 2-methylpropanoate | 2.3 | Combines with MO | Negative | B |
| | | but propionic off | | |
| | | note | | |
| 2-phenylethyl acetate | 1 | No effect when | Negative | B |
| | | used alone | | |
| 2-phenylethanol | 3.2 | Combines with MO | Negative | B |
| | | but propionic off | | |
| | | note | | |
| 2,4-dimethyl-4-phenyltetrahydrofuran | 4.1 | Very performing, | Negative | B |
| | | but unpleasant | | |
| | | animalic off-note | | |
| cyclopentadecanone, hexadecanolide | 1 | No effect when | Negative | B |
| | | used alone | | |
| Spearmint oil | 2 | Low coverage | Negative | B |
| ethyl methyl phenyl glycidate | 1 | No effect when | Negative | B |
| | | used alone | | |
| (E)-2-((3,5-dimethylhex-3-en-2- | 1 | No effect when | Negative | B |
| yl)oxy)-2-methylpropyl | | used alone | | |
| cyclopropanecarboxylate | | | | |
| 3,7-dimethyloctan-3-ol | 3 | Good coverage of | Negative | B |
| | | MO but plastic off- | | |
| | | note | | |
| 2,4-dimethylcyclohex-3- | 3.8 | Good coverage | Negative | B |
| enecarbaldehyde | | | | |
| (Z)-cyclohexadec-5-enone | 1 | Enhances MO | Negative | B |

1. A scalp malodour counteracting fragrance composition comprising one or more fragrance ingredients drawn from Group A and one or more fragrance ingredients from Group B, wherein the sum of the individual concentrations of all fragrance ingredients drawn from Group A is 10 wt. % or more, based on the total weight of the scalp malodour counteracting fragrance composition; wherein the sum of the individual concentrations of all fragrance ingredients from Group B is 0.1 wt. % or more, based on the total weight of the scalp malodour counteracting fragrance composition; and wherein the weight ratio of the sum of the individual concentrations of all fragrance ingredients drawn from Group A to the sum of the individual concentrations of all fragrance ingredients drawn from Group B is 0.8 or more;
1 wherein the Group A fragrance ingredients are selected from the group consisting of 3-hydroxybutan-2-one; 1-phenylethanone; 2-(tert-butyl)cyclohexyl acetate; 2-methyl-undecanal; 1-((2-(tert-butyl)cyclohexyl)oxy) butan-2-ol; (4aR,5R,7aS,9R)-octahydro-2,2,5,8,8,9a-hexamethyl-4H-4a,9-methanoazuleno [5,6-d]-1,3-dioxole; (3aR,5aS,9aS, 9bR)-3a,6,6,9a-tetramethyl-2,4,5,5a,7,8,9,9b-octahydro-1H-benzo[e][1]benzofuran; (E)-1-methoxy-4-(prop-1-en-1-yl)benzene; anise and star anise oils; 4-methoxybenzaldehyde; 7-isopentyl-2H-benzo[b][1,4]dioxepin-3 (4H)-one; benzoin oils; 1-butoxy-1-oxopropan-2-yl butanoate; 8-methyl-1,5-benzodioxepin-3-one; 6-methoxy-2,6-dimethyloctanal; (1S,4S)-1,7,7-trimethylbicyclo[2.2.1]heptan-2-one; blackcurrant oils and reconstitutions; 5-tert-butyl-2-methyl-5-propyl-2H-furan; cedar wood oils; (1S,6R,8aR)-1,4,4,6-tetramethyloctahydro-1H-5,8a-methanoazulen-6-yl acetate; 3,7-dimethyloct-6-enal; 3,7-dimethyloct-6-en-1-ol; (4-isopropyl-phenyl) methanol; cyclohexyl 2-hydroxybenzoate; (E)-1-(2,6,6-trimethylcyclohexa-1,3-dien-1-yl) but-2-en-1-one; (E)-1-(2,6,6-trimethylcyclohex-2-en-1-yl) but-2-en-1-one; (E)-1-(2,6,6-trimethyl-1-cyclohexenyl) but-2-en-1-one; 5-hexyloxolan-2-one; 2-methoxy-4-propylphenol; 6-heptyltetrahydro-2H-pyran-2-one; (E)-4-((3aS,7aS)-hexahydro-1H-4,7-methanoinden-5 (6H)-ylidene) butanal; (E)-3-methyl-5-(2,2,3-tri-methylcyclopent-3-en-1-yl) pent-4-en-2-ol; ethyl 2-methyl-propanoate; ethyl octanoate; ethyl heptanoate; 4-allyl-2-methoxyphenol; 3-(4-methoxyphenyl)-2-methylpropanal; 1-(3,5,5,6,8,8-hexamethyl-5,6,7,8-tetrahydro-naphthalen-2-yl) ethanone; ethyl 2-(2-methyl-1,3-dioxolan-2-yl)acetate; 4,6,6,7,8,8-hexamethyl-1,3,4,6,7,8-hexahydro-cyclopenta [g] isochromene; geranium oils; benzo[d][1,3]dioxole-5-carbaldehyde; 7-hydroxy-3,7-dimethyloctanal; 3,7-dimethyloctane-1,7-diol; 1H-indole; 1-(2,3,8,8-tetramethyl-1,2,3,4,5,6,7,8-octahydronaphthalen-2-yl) ethanone; 4-formyl-2-methoxy-phenyl 2-methylpropanoate; (E)-2-methoxy-4-(prop-1-en-1-yl) phenol; (Z)-1-(2,6,6-trimethylcyclohex-2-en-1-yl) pent-1-en-3-one, (E)-1-(2,6,6-trimethylcyclohex-2-en-1-yl) pent-1-en-3-one and mixtures thereof; (E)-3-methyl-4-(2,6,6-trimethylcyclohex-2-en-1-yl) but-3-en-2-one; (1-methyl-2-((1,2,2-trimethylbicyclo[3.1.0]hexan-3-yl)methyl)cyclopropyl) methanol; 3-(4-(tert-butyl)phenyl)-2-methylpropanal; (4E)-9-hydroxy-5,9-dimethyl-4-decenal; 3-hydroxy-2-methyl-4H-pyran-4-one; (4-iso-propylcyclohexyl) methanol; 3-methyl-5-phenylpentan-1-ol; 2-isopropyl-5-methyl-cyclohexanol; mercapto-para-menthan-3-one; methyl 2-aminobenzoate; 8-methyl-1-oxaspiro[4.5]decan-2-one; 8-ethyl-1-oxaspiro[4.5]decan-2-one methyl non-2-ynoate; (E)-3-methyl-4-(2,6,6-trimethylcyclohex-1-en-1-yl) but-3-en-2-one; (Z)-3-methylcyclo-penta-dec-5-enone; (E)-methyl non-2-enoate; (2Z)-3,7-dimethylocta-2,6-dien-1-ol; (E)-3,7, 11-trimethyldodeca-1,6,10-trien-3-ol; (E)-13-methyloxacyclopentadec-10-en-2-one; 2-(6,6-dimethylbicyclo[3.1.1]hept-2-en-2-yl)ethyl acetate; 3-(4-(2-methylpropyl)-2-methyl-phenyl) propanal; patchouli oils; 3,7-dimethyloctan-1-ol; (2E,5E)-5,6,7-trimethylocta-2,5-dien-4-one; (2R,8aS)-3′,6-dimethyl-3,4,4a,5,8,8a-hexahydro-1H-spiro[1,4-methano-naphthalene-2,2′-oxirane]; 1-methyl-2-(5-methylhex-4-en-2-yl)cyclopropylmethanol; 2-(4-methyl-1,3-thiazol-5-yl) ethanol; (E)-6-ethyl-3-methyloct-6-en-1-ol; 1-((2E,5Z,9Z)-2,7,8-trimethylcyclododeca-2,5,9-trien-1-yl) ethanone; 2-ethoxy-4-methylphenol; 2,2,5-trimethyl-5-pentylcyclopentanone; 4,4a-dimethyl-6-(prop-1-en-2-yl)-4,4a,5,6,7,8-hexa-hydronaphthalen-2 (3H)-one; 4a,5-dimethyl-3-prop-1-en-2-yl-2,3,4,5,6,7-hexahydro-1H-naphthalene; (4R,4aS,6S)-4,4a-dimethyl-6-prop-1-en-2-yl-3,4,5,6-tetrahydronaphthalen-2-one; and mixtures thereof; and 1 wherein the Group B fragrance ingredients are selected from the group consisting of (2-benzyl-1,3-dioxolan-4-yl) methanol; undecanal; prop-2-enyl 3-cyclohexyl-propanoate; pineapple oils and reconstitutions; benzyl acetate; benzyl propanoate; (5R)-2-methyl-5-prop-1-en-2-ylcyclohex-2-en-1-one; (E)-1-(2,6,6-trimethylcyclohex-2-en-1-yl) hepta-1,6-dien-3-one; 3,7-dimethyloct-6-en-1-yl acetate; 2,4-dimethylcyclo-hex-3-ene-1-carbaldehyde; allyl 2-(cyclohexyloxy)acetate; 4-(2,6,6-trimethylcyclohex-1-en-1-yl) butan-2-one; 2-methyl-1-phenylpropan-2-ol; 2-methyl-1-phenylpropan-2-yl acetate; 2-methyl-1-phenylpropan-2-yl butanoate; 4,7-dimethyloct-6-en-3-one; (2E,4Z)-ethyl deca-2,4-dienoate; ethyl 2,6,6-trimethylcyclohexa-1,3-diene-1-carboxylate; 4-allyl-2-methoxy-phenyl acetate; (3aR,6S,7aS)-3a,4,5,6,7,7a-hexa-hydro-1H-4,7-methanoinden-6-yl propanoate; 2,4,6-trimethyl-4-phenyl-1,3-dioxane; tetrahydro-4-methyl-2-(2-methylpropyl)-2H-pyran-4-ol; tetrahydro-4-methyl-2-(2-methylpropyl)-2H-pyran-4-ol; 2-ethyl-4-hydroxy-5-methylfuran-3 (2H)-one; (Z)-hex-3-en-1-yl benzoate; (E)-2-benzylideneoctanal; (E)-4-(2,6,6-trimethylcyclohex-1-en-1-yl) but-3-en-2-one; (E)-4-(2,6,6-trimethyl-1-cyclohex-2-enyl) but-3-en-2-one; (3aR,6S, 7aS)-3a,4,5,6,7,7a-hexahydro-1H-4,7-methanoinden-6-yl acetate; (Z)-3-methyl-2-(pent-2-en-1-yl)cyclopent-2-enone; 3-butyl-5-methyltetrahydro-2H-pyran-4-yl acetate; lemongrass oils; lime oils; 1-((1S,8aS)-1,4,4,6-tetramethyl-2,3,3a,4,5,8-hexahydro-1H-5,8a-methanoazulen-7-yl) ethanone; methyl 2-hexyl-3-oxocyclopentane-1-carboxylate; (E)-1,2-dimethoxy-4-(prop-1-en-1-yl)benzene; 4-(4-methylpent-3-en-1-yl)cyclohex-3-enecarbaldehyde; (Z)-3,7-dimethylocta-2,6-dien-1-yl acetate; 6-propyltetrahydro-2H-pyran-2-one; 5-butyloxolan-2-one; blood orange oils; orange oils; bitter orange oils; orange terpenes; sweet orange oils; 1-(2-naphtalenyl)-ethanone; 2-methyl-4-propyl-1,3-oxathiane; passion fruit oils; 2-cyclohexylidene-2-phenylacetonitrile; 2-(phenoxy)ethyl 2-methylpropanoate; 2-phenylethyl acetate; 2-phenylethanol; 2,4-dimethyl-4-phenyltetrahydrofuran; cyclopentadecanone, hexadecanolide; spearmint oils; ethyl methyl phenyl glycidate; (E)-2-((3,5-dimethylhex-3-en-2-yl)oxy)-2-methylpropyl cyclopropanecarboxylate; 3,7-dimethyloctan-3-ol; 2,4-dimethylcyclohex-3-enecarbaldehyde; (Z)-cyclohexadec-5-enone; and mixtures thereof.

2. The scalp malodour counteracting fragrance composition according to claim 1, comprising, based on the total weight of the scalp malodour counteracting fragrance composition, at least 8 wt. %, of at least 1, 3 Group A fragrance ingredients from Group A1, wherein the Group A1 fragrance ingredients are selected from the group consisting of 2-(tert-butyl)cyclohexyl acetate; 4-methoxybenzaldehyde; benzoin oils; 3,7-dimethyloct-6-en-1-ol; 5-hexyloxolan-2-one; ethyl 2-(2-methyl-1,3-dioxolan-2-yl)acetate; 4,6,6,7,8,8-hexamethyl-1,3,4,6,7,8-hexahydrocyclo-penta [g] isochromene; benzo[d][1,3]dioxole-5-carbaldehyde; 7-hydroxy-3,7-dimethyloctanal; 3,7-dimethyloctane-1,7-diol; 1-(2,3,8,8-tetramethyl-1,2,3,4,5,6,7,8-octahydronaphthalen-2-yl) ethanone; (E)-3-methyl-4-(2,6,6-trimethyl-2-cyclohexen-1-yl)-3-buten-2-one; (Z)-1-(2,6,6-tri-methylcyclohex-2-en-1-yl) pent-1-en-3-one, (E)-1-(2,6,6-trimethylcyclohex-2-en-1-yl) pent-1-en-3-one and mixtures thereof; 3-(4-(tert-butyl)phenyl)-2-methylpropanal; (4-isopropyl-cyclohexyl) methanol; 2-(6,6-dimethylbicyclo[3.1.1]hept-2-en-2-yl)ethyl acetate; 3-(4-(2-methyl-propyl)-2-methylphenyl) propanal; 1-methyl-2-(5-methylhex-4-en-2-yl)cyclopropyl-methanol; 1-((2E,5Z,9Z)-2,7,8-trimethylcyclododeca-2,5,9-trien-1-yl) ethanone; and mixtures thereof.

3. The scalp malodour counteracting fragrance composition according to claim 1, comprising, based on the total weight of the scalp malodour counteracting fragrance composition, from 0.3 wt. % to 10 wt. %, of at least 1, Group A fragrance ingredients from Group A2, wherein the Group A2 fragrance ingredients are selected from the group consisting of 3-hydroxybutan-2-one; 2-methylundecanal; 1-((2-(tert-butyl)cyclohexyl)oxy) butan-2-ol; (3aR,5aS,9aS,9bR)-3a,6,6,9a-tetramethyl-2,4,5,5a,7,8,9,9b-octahydro-1H-benzo[e][1]benzofuran; (E)-1-methoxy-4-(prop-1-en-1-yl)benzene; benzoin oils; 1-butoxy-1-oxopropan-2-yl butanoate; 8-methyl-1,5-benzodioxepin-3-one; cedar wood oils; (1S,6R,8aR)-1,4,4,6-tetramethyloctahydro-1H-5,8a-methanoazulen-6-yl acetate; (E)-1-(2,6,6-trimethylcyclohexa-1,3-dien-1-yl) but-2-en-1-one; (E)-1-(2,6,6-trimethylcyclohex-2-en-1-yl) but-2-en-1-one; 6-heptyltetrahydro-2H-pyran-2-one; (E)-4-((3aS,7aS)-hexahydro-1H-4,7-methanoinden-5 (6H)-ylidene) butanal; (E)-3-methyl-5-(2,2,3-trimethylcyclopent-3-en-1-yl) pent-4-en-2-ol; ethyl 2-methylpropanoate; 3-(4-methoxyphenyl)-2-methylpropanal; 1-(3,5,5,6,8,8-hexamethyl-5,6,7,8-tetrahydronaphthalen-2-yl) ethanone; geranium oils; 4-formyl-2-methoxyphenyl 2-methylpropanoate; (Z)-3-methyl-4-(2,6,6-trimethyl-2-cyclohexen-1-yl)-3-buten-2-one; (E)-1-(2,6,6-trimethylcyclohex-2-en-1-yl) pent-1-en-3-one; (E)-3-methyl-4-(2,6,6-trimethylcyclohex-2-en-1-yl) but-3-en-2-one; (4E)-9-hydroxy-5,9-dimethyl-4-decenal; 3-hydroxy-2-methyl-4H-pyran-4-one; 3-methyl-5-phenylpentan-1-ol; 2-isopropyl-5-methylcyclohexanol; methyl 2-aminobenzoate; 8-methyl-1-oxaspiro[4.5]decan-2-one; 8-ethyl-1-oxaspiro[4.5]decan-2-one; (E)-3-methyl-4-(2,6,6-trimethylcyclohex-1-en-1-yl) but-3-en-2-one; (Z)-3-methylcyclopentadec-5-enone; (E)-13-methyloxacyclopentadec-10-en-2-one; patchouli oils; 3,7-dimethyloctan-1-ol; (2R,8aS)-3′,6-dimethyl-3,4,4a,5,8,8a-hexahydro-1H-spiro[1,4-methanonaphthalene-2,2′-oxirane]; and mixtures thereof.

4. The scalp malodour counteracting fragrance composition according to claim 1, comprising, based on the total weight of the malodour counteracting fragrance composition, from 0.05 wt. % to 2 wt. %, of at least 1, Group A fragrance ingredients from Group A3, wherein the Group A3 fragrance ingredients are selected from the group consisting of (4aR,5R,7aS,9R)-octahydro-2,2,5,8,8,9a-hexamethyl-4H-4a,9-methanoazuleno [5,6-d]-1,3-dioxole; anise and star anise oils; 7-isopentyl-2H-benzo[b][1,4]dioxepin-3 (4H)-one; 6-methoxy-2,6-dimethyloctanal; (1S,4S)-1,7,7-trimethyl-bicyclo[2.2.1]heptan-2-one; blackcurrant oils and reconstitutions; 5-tert-butyl-2-methyl-5-propyl-2H-furan; (1S,6R,8aR)-1,4,4,6-tetramethyloctahydro-1H-5,8a-methanoazulen-6-yl acetate; 3,7-dimethyloct-6-enal; (4-isopropylphenyl) methanol; cyclo-hexyl 2-hydroxybenzoate; (E)-1-(2,6,6-trimethyl-1-cyclohexenyl) but-2-en-1-one; 2-methoxy-4-propylphenol; ethyl octanoate; ethyl heptanoate; 4-allyl-2-methoxyphenol; 1H-indole; (E)-2-methoxy-4-(prop-1-en-1-yl) phenol; (1-methyl-2-((1,2,2-trimethylbicyclo[3.1.0]hexan-3-yl)methyl)cyclopropyl) methanol; mercapto-para-menthan-3-one; 8-methyl-1-oxaspiro[4.5]decan-2-one; methyl non-2-ynoate; (E)-methyl non-2-enoate; (2Z)-3,7-dimethylocta-2,6-dien-1-ol; (E)-3,7,11-trimethyldodeca-1,6, 10-trien-3-ol; (2E,5E)-5,6,7-tri-methylocta-2,5-dien-4-one; 2-(4-methyl-1,3-thiazol-5-yl) ethanol; (E)-6-ethyl-3-methyloct-6-en-1-ol; 2-ethoxy-4-methylphenol; 2,2,5-trimethyl-5-pentylcyclopentanone; and mixtures thereof.

5. The scalp malodour counteracting fragrance composition according to claim 1, comprising at least one fragrance ingredient from at least two groups selected from Groups A1, A2, and A3.

6. The scalp malodour counteracting fragrance composition according to claim 5, comprising at least one fragrance ingredient from each of A1, A2 and A3, wherein the amounts of Groups A1, A2, A3 and B ingredients fulfil the inequality defined by Equation 1:
$$\frac{{W}_{A 1} + 10 {W}_{A 2} + 100 {W}_{A 3}}{{W}_{B}} > 2 \qquad Equation 1$$
wherein W_{A1} is the sum of the individual concentrations of all fragrance ingredients drawn from Group A1 fragrance ingredients expressed in wt. %, W_{A2} is the sum of the individual concentrations of all fragrance ingredients drawn from Group A2 fragrance ingredients expressed in wt. %, W_{A3} is the sum of the individual concentrations of all fragrance ingredients from Group A3 fragrance ingredients expressed in wt. %, and W_{B} is the amount of Group B fragrance ingredients, all weight percentage wt. % being based on the total weight of the scalp malodour counteracting fragrance composition.

7. The scalp malodour counteracting fragrance composition according claim 1 comprising
1-(2,3,8,8-tetramethyl-1,2,3,4,5,6,7,8-octahydronaphthalen-2-yl) ethanone;
at least one further woody fragrance ingredient selected from the group consisting of (1-methyl-2-((1,2,2-trimethylbicyclo[3.1.0]hexan-3-yl)methyl)cyclopropyl)-methanol, (E)-3-methyl-5-(2,2,3-trimethylcyclopent-3-en-1-yl) pent-4-en-2-ol, cedar wood oil, and mixtures thereof;
at least one lactone selected from the group consisting of 5-hexyloxolan-2-one; 6-pentyltetrahydro-2H-pyran-2-one, 5-octyloxolan-2-one, 8-isopropyl-1-oxaspiro[4.5]decan-2-one, 8-methyl-1-oxaspiro[4.5]decan-2-one, 8-ethyl-1-oxaspiro[4.5]decan-2-one, and mixtures thereof;
at least one floral fragrance ingredient selected from the group consisting of (E)-3-methyl-4-(2,6,6-trimethylcyclohex-2-en-1-yl) but-3-en-2-one, (E)-4-((3aS,7aS)-hexa-hydro-1H-4,7-methanoinden-5 (6H)-ylidene) butanal, 3-(4-(2-methylpropyl)-2-methyl-phenyl) propa-nal, 3-methyl-5-phenylpentan-1-ol, tetrahydro-4-methyl-2-(2-methyl-propyl)-2H-pyran-4-ol, 1-methyl-2-(5-methylhex-4-en-2-yl)cyclopropylmethanol, and mixtures thereof; and
at least one sweet fragrance ingredient selected from benzo[d][1,3]dioxole-5-carbaldehyde, ethyl 2-(2-methyl-1,3-dioxolan-2-yl)acetate, 3-hydroxy-2-8.

8. The scalp malodour counteracting fragrance composition according claim 1 comprising
at least 1 wt. % of 3,7-dimethyloct-6-en-1-ol, 5-hexyloxolan-2-one, or a mixture re-thereof; and
at least 1, fragrance ingredient selected from the group consisting of 1-phenylethanone, (3aR,5aS,9aS,9bR)-3a,6,6,9a-tetramethyl-2,4,5,5a,7,8,9,9b-octahydro-1H-benzo[e][1]benzofuran, (E)-1-methoxy-4-(prop-1-en-1-yl)benzene, 3-(4-methoxyphenyl)-2-methylpropanal, 5-tert-butyl-2-methyl-5-propyl-2H-furan, (E)-1-(2,6,6-trimethylcyclohexa-1,3-dien-1-yl) but-2-en-1-one, (E)-1-(2,6,6-trimethylcyclohex-2-en-1-yl) but-2-en-1-one, (E)-1-(2,6,6-trimethyl-1-cyclohexenyl) but-2-en-1-one, (E)-2-methoxy-4-(prop-1-en-1-yl) phenol, 2-methoxy-4-propylphenol, 4-allyl-2-methoxyphenol, (2R,8aS)-3′,6-dimethyl-3,4,4a,5,8,8a-hexahydro-1H-spiro[1,4-methanonaphthalene-2,2′-oxirane], anise or star anise oils, and blackcurrant oils and reconstitutions, and mixtures thereof.

9. The scalp malodour counteracting fragrance composition according claim 1, comprising from 15 to 60 wt. %, of at least 1, fragrance ingredient selected from the group consisting of (E)-2-methoxy-4-(prop-1-en-1-yl)phenyl acetate; 2,6,10-trimethylundec-9-enal; tridecan-1-ol; decanal; 2-methyldecanal; undec-10-enal; dodecanal; hexan-1-al; octanal; 3,5,5-trimethylhexanal; nonanal; (E)-dodec-2-enal; prop-2-enyl 2-(3-methylbutoxy)acetate; prop-2-enyl hexanoate; prop-2-enyl heptanoate; decahydro-2,2,6,6,7,8,8-heptamethyl indenofuran; 3,8,8,11a-tetramethyldodecahydro-1H-3,5a-epoxynaphtho[2,1-c] oxepine; 2-(2,2,7,7-tetra-methyltricyclo[6.2.1.0] (1,6) undec-4-en-5-yl) propan-1-ol, 2-(2,2,7,7-tetramethyltricyclo[6.2.1.0] (1,6) undec-5-en-5-yl) propan-1-ol and mixtures thereof; (E)-methyl 2-((7-hydroxy-3,7-dimethyloctylidene)amino)benzoate; benzyl 3-phenylprop-2-enoate; benzyl 2-hydroxy-benzoate; octahydro-2H-chromen-2-one; (1S,2S,4S)-1,7,7-trimethylbicyclo[2.2.1]heptan-2-ol; 3-(4-(tert-butyl)phenyl) propanal; 4-(tert-butyl)cyclohexyl acetate; 8-methyl-1,5-benzodioxepin-3-one; 1,1,2,3,3-pentamethyl-2,3,6,7-tetrahydro-1H-inden-4 (5H)-one; (E)-3-phenylprop-2-en-1-ol; (E)-3,7-dimethylocta-2,6-dienal; 3,7-dimethyloct-6-enenitrile; 2-hydroxy-3-methylcyclopent-2-enone; 2H-chromen-2-one; 4-isopropylbenzaldehyde; 3-(4-isopropylphenyl)-2-methylpropanal; 8,8-dimethyl-1,2,3,4,5,6,7,8-octahydronaphthalene-2-carbaldehyde; (E)-1-(2,6,6-trimethyl-cyclohexa-1,3-dien-1-yl) but-2-en-1-one; 1-(2,6,6-trimethyl-1-cyclohex-3-enyl) but-2-en-1-one; 2,6-dimethyloct-7-en-2-ol; 2,6-dimethylheptan-2-ol; (E)-dodec-2-enal; ethyl benzoate; 8-ethyl-1-oxaspiro[4.5]decan-2-one; (E)-3,7-dimethylnona-1,6-dien-3-ol; 2-ethyl-3-hydroxy-4H-pyran-4-one; ethyl 2-methylbutanoate; 1,4-dioxacycloheptadecane-5, 17-dione; (1S,4S)-1,3,3-trimethyl-2-oxabicyclo[2.2.2]octane; 3-(4-ethylphenyl)-2,2-dimethylpropanal; 3-(3-iso-propylphenyl) butanal; 1-(5,5-dimethylcyclohex-1-en-1-yl) pent-4-en-1-one; (E)-3,7-dimethyl-octa-2,6-dien-1-ol; (E)-3,7-dimethylocta-2,6-dien-1-yl acetate; (E)-3,7-dimethylocta-2,6-dien-1-yl formate; (E)-hex-2-enal; (E)-hex-2-en-1-ol; (Z)-hex-3-en-1-ol; (Z)-hex-3-en-1-yl acetate; (Z)-hex-3-en-1-yl 2-hydroxybenzoate; hexyl acetate; (1,1-dimethoxypropan-2-yl)benzene; (4Z)-hept-4-en-2-yl 2-hydroxybenzoate; (Z)-hex-3-en-1-yl methyl carbonate; 2,2,6-trimethyl-6-vinyltetrahydro-2H-pyran; 3,7-dimethylocta-1,6-dien-3-ol; 3,7-dimethyl-octa-1,6-dien-3-yl acetate; 3,7-dimethylocta-1,6-dien-3-yl 2-methylpropanoate; bicyclo[2.2.2]oct-5-ene-2-carboxaldehyde; 2,2-dimethyl-3-(m-tolyl) propan-1-ol; ethyl 2-methyl-pentanoate; 3-methyl-5-phenylpentanal; 2,6-dimethylhept-5-enal; methyl benzoate; methyl 2-methylbutanoate; (2E,6Z)-nona-2,6-dienal; (2Z,6E)-2,6-nonadien-1-ol; (Z)-non-6-en-1-ol; 2-cyclohexylhepta-1,6-dien-3-one; 2-phenoxyethanol; 3-phenylpropan-1-ol; 3-methylbut-2-en-1-yl acetate; 5-pentyldihydrofuran-2 (3H)-one; (E)-2-ethyl-4-(2,2,3-trimethylcyclopent-3-en-1-yl) but-2-en-1-ol; (E)-2-ethyl-4-(2,2,3-trimethylcyclopent-3-en-1-yl) but-2-en-1-ol; 4-(4-hydroxyphenyl) butan-2-one; 4-methyl-2-(2-methylprop-1-en-1-yl)tetrahydro-2H-pyran; 4-methyl-2-(2-methylprop-1-en-1-yl)tetrahydro-2H-pyran; (3aR,4R,6S,7R,7aR)-6-methoxy-octahydro-1H-4,7-methanoindene-1-carbaldehyde; 2-methyl-3-[4-(2-methylpropyl)phenyl] propanal; 1-(spiro[4.5]dec-6-en-7-yl) pent-4-en-1-one; 1-phenylethyl acetate; 2-(4-methyl-1-cyclohex-3-enyl) propan-2-ol; 2-(4-methyl-1-cyclohex-3-enyl) propan-2-yl acetate; oxacyclo-hexadecan-2-one; 2-isopropyl-5-methylphenol; 3-(benzo[d][1,3]dioxol-5-yl)-2-methylpro-panal; (E)-4-methyldec-3-en-5-ol; 3,4-dimethoxybenzaldehyde; (E)-methyl 2-((3-(4-(tert-butyl)phenyl)-2-methylprop-1-en-1-yl)amino)benzoate; 1-methyl-4-(4-methylpentyl)cyclo-hex-3-enecarbaldehyde; (2E,6Z)-nona-2,6-dienenitrile; and mixtures thereof.

10. The scalp malodour counteracting fragrance composition according claim 1 further comprising from 0.1 wt. % to 50 wt. %, of a solvent selected from the group consisting of propylene glycol, dipropylene glycol, triethyl citrate, diethyl citrate, isopropylidene glycerol, 1,2-propane diol, 1,3-propane diol, 1,2-butane diol, 1,3-butane diol, 1,2-pentane diol, triacetine, dimethyl isosorbide, dimethyl carbonate, isoparaffinic hydrocarbons, and mixtures thereof.

11. A method of assessing the scalp malodour counteraction capability of a test fragrance composition, the method comprising the steps of:
a. Applying a product composition comprising the test fragrance composition onto a clean fabric;
b. Removing the product composition by rinsing the fabric with water;
c. Letting the fabric dry in air;
d. Placing the fabric into a confined space, in the presence of a scalp malodour;
e. Closing the confined space;
f. Opening the confined space after equilibration and assessing both malodour intensity and fragrance intensity in this confined space;
wherein a scalp malodour counteraction capability is confirmed if the fragrance intensity is stronger than the malodour intensity.

12. A cosmetic product comprising the scalp malodour counteracting fragrance composition according to claim 1 and a suitable carrier, wherein the cosmetic product.

13. The cosmetic product according to claim 12, wherein the concentration of the malodour counteracting fragrance composition in the cosmetic product is from 0.1 wt. % to 5 wt. %, based on the total weight of the cosmetic product.

14. The cosmetic product according to claim 11 further comprising one or more of skin care agents, soothing agents, deodorizing agent, probiotics, prebiotics, emollients, surfactants, thickening agents, sensates, polyols, humectants, polymers, silicones, enzymes, inorganic fillers, cationics, amphoterics, glycerides, esters, ethers, amino acids, polysaccharides, sugars, water or combinations thereof.

15. A method of masking, reducing and/or preventing scalp malodour, said method comprising the step of applying the scalp malodour counteracting fragrance composition according to claim 1 to the human scalp or hair.

16. A method of assessing the scalp malodour counteraction capability of a test fragrance composition, wherein the said test fragrance composition is a scalp malodour counteracting fragrance composition of claim 1, the method comprising the steps of:
a. Applying a product composition comprising the test fragrance composition onto a clean fabric;
b. Removing the product composition by rinsing the fabric with water;
c. Letting the fabric dry in air;
d. Placing the fabric into a confined space, in the presence of a scalp malodour;
e. Closing the confined space;
f. Opening the confined space after equilibration and assessing both malodour intensity and fragrance intensity in this confined space;
wherein a scalp malodour counteraction capability is confirmed if the fragrance intensity is stronger than the malodour intensity.
